(19) United States
(12) Patent Application Publication
(10) Pub. No.: US 2023/0391871 A1
(43) Pub. Date: Dec. 7, 2023

(54) MONOAMINE OXIDASE BLOCKADE THERAPY FOR TREATING CANCER THROUGH REGULATING TUMOR ASSOCIATED MACROPHAGES (TAMS)

(71) Applicant: THE REGENTS OF THE UNIVERSITY OF CALIFORNIA, OAKLAND, CA (US)

(72) Inventors: Lili Yang, Los Angeles, CA (US); Xi Wang, Los Angeles, CA (US); Yuchen Wang, Los Angeles, CA (US)

(73) Assignee: THE REGENTS OF THE UNIVERSITY OF CALIFORNIA, OAKLAND, CA (US)

(21) Appl. No.: 18/249,907

(22) PCT Filed: Oct. 22, 2021
(86) PCT No.: PCT/US21/56269

§ 371 Date: Apr. 20, 2023

Related U.S. Application Data
(60) Provisional application No. 63/104,174, filed on Oct. 22, 2020.

(51) Int. Cl. C07K 16/28; A61P 35/00; A61K 45/06; A61K 47/69

(52) U.S. Cl. CPC C07K 16/2818; C07K 16/2827; A61P 35/00; A61K 45/06; A61K 47/6929

(57) ABSTRACT

We have discovered MAO-A induction in mouse and human TAMs. Moreover, we determined that MAO-A-deficient mice exhibited decreased TAM immunosuppressive functions corresponding with enhanced antitumour immunity. Building upon these discoveries, we then determined that MAOI treatment induced TAM reprogramming and suppressed tumour growth in preclinical mouse syngeneic and human xenograft tumour models. Surprisingly, combining MAOI and anti-PD-1 treatments resulted in synergistic tumour suppression. Together, these data identify MAO-A as a critical regulator of TAMs, and that show that repurposing MAOIs for TAM reprogramming can be used to improve cancer immunotherapies.

### CROSS-REFERENCE TO RELATED APPLICATIONS

This application claims the benefit under 35 U.S.C. Section 119(e) of co-pending and commonly-assigned U.S. Provisional Patent Application Ser. No. 63/104,174, filed on Oct. 22, 2020, and entitled “MONOAMINE OXIDASE BLOCKADE THERAPY FOR TREATING CANCER THROUGH REGULATING TUMOR ASSOCIATED MACROPHAGES (TAMS)” which application is incorporated by reference herein.

### STATEMENT REGARDING FEDERALLY SPONSORED RESEARCH AND DEVELOPMENT

This invention was made with government support under Grant Number CA196335, awarded by the National Institutes of Health. The government has certain rights in the invention.

### TECHNICAL FIELD

The present invention relates to methods and materials for treating cancers.

### BACKGROUND OF THE INVENTION

Over the past decade, cancer immunotherapy has achieved significant breakthroughs. In particular, immune checkpoint blockade (ICB) therapy has yielded remarkable clinical responses and revolutionized the treatment of many cancers¹. So far, the FDA has approved cytotoxic T-lymphocyte antigen 4 (CTLA-4) and programmed cell death protein 1/ligand 1 (PD-1/PD-L1) blockade therapies for treating more than 10 different malignancies^{2, 3}; however, only a small fraction of cancer patients respond to these therapies^{4, 5, 6}. Most ICB therapies work through enhancing antitumour CD8⁺ T cell responses, which can be greatly limited by the immunosuppressive tumour microenvironment (TME)⁷. Tumour-associated macrophages (TAMs), a key component of the immunosuppressive TME, dampen T cell antitumour reactivity in the majority of solid tumours^{8, 9, 10, 11, 12, 13, 14, 15}. Growing evidence suggests that TAMs are responsible for inhibiting antitumour T cell reactivity and limiting the ICB therapy efficacy, making TAMs potential targets for reversing the immunosuppressive TME and improving cancer immunotherapy^{16, 17, 18}.

TAMs mature from bone marrow-derived circulating monocytes. These monocytes are recruited to the tumour sites, exposed to chemokines and growth factors in the TME, and subsequently differentiate into TAMs^{19, 20, 21, 22}. Depending on the surrounding immune environment, macrophages can be polarized toward an immunostimulatory phenotype by pro-inflammatory stimuli (e.g., IFN-γ) or toward an immunosuppressive phenotype by anti-inflammatory stimuli (e.g., IL-4 and IL-13)²³. Although a binary polarization system is commonly used in macrophage studies, in most large-scale transcriptome analyses, TAMs showed a continuum of phenotypes expressing both immunostimulatory and immunosuppressive markers in addition to the extreme ends of polarization^{23, 24, 25}. These mixed phenotypes and polarization states suggest the complexity of the TME and the residential TAM functionality. As a tumour develops, the enrichment of IL-4 and IL-13 produced by tumour cells and CD4⁺ T cells in the TME results in the polarization of TAMs towards an immunosuppressive phenotype, that promotes tumour growth, malignancy, and metastasis^{23, 26, 27, 28, 29, 30}. In established solid tumours, TAMs predominately exhibit an immunosuppressive phenotype, evidenced by their production of anti-inflammatory cytokines and arginase-1 (Arg1), as well as their expression of mannose receptor (CD206) and scavenger receptors^{31, 32, 33}. Through metabolizing L-arginine via Arg1, TAMs can directly suppress cytotoxic CD8⁺ T cell responses^{34, 35}. Mannose receptor (CD206) expressed by TAMs can impair cytotoxicity of CD8⁺ T cells by suppressing CD45 phosphatase activity³⁶. In addition, TAMs can inhibit T cell activities through immune checkpoint engagement by expressing the ligands of the inhibitory receptors PD-1 and CTLA-4. For example, PD-L1 and PD-L2 expressed on TAMs interact with PD-1 of T cells to directly inhibit TCR signaling, cytotoxic function, and proliferation of CD8⁺ T cells³¹. These characteristics of TAMs make them potential targets for reversing the immunosuppressive TME to augment antitumour immunity.

Although the predominant phenotype of TAMs in established solid tumours is immunosuppressive, polarization is not fixed. Plasticity, one of the key features of TAMs, enables TAMs to change their phenotype in solid tumours and thereby providing a therapeutic window^{37, 38}. Repolarizing/reprogramming TAMs from an immunosuppressive and tumour-promoting phenotype toward an immunostimulatory and tumouricidal phenotype has thus become an attractive strategy in immunotherapy²⁷. Preclinical and clinical studies are ongoing, evaluating TAM-repolarizing reagents (e.g., CD40 agonists, HDAC inhibitors, PI3Kγ inhibitors, creatine, etc.) for improving ICB therapy: certain efficacies have been reported^{17, 29, 31, 39, 40, 41, 42}. Therefore, the search for new molecules regulating TAM polarization and the development of new combination treatments targeting TAM reprogramming are an active direction of current cancer immunotherapy studies.

For the reasons noted above, there is a need in the art for the development of methods and materials for regulating TAM polarization in cancer immunotherapies.

### SUMMARY OF THE INVENTION

Targeting tumour-associated macrophages (TAMs) is a promising strategy to modify the immunosuppressive tumour microenvironment and improve cancer immunotherapy. Monoamine oxidase A (MAO-A) is an enzyme best known for its function in the brain: small molecule MAO inhibitors (MAOIs) are clinically used for treating neurological disorders. As discussed in detail below, we have discovered MAO-A induction in mouse and human TAMs. Moreover, we determined that MAO-A-deficient mice exhibited decreased TAM immunosuppressive functions corresponding with enhanced antitumour immunity. Building upon these discoveries, we then determined that MAOI treatment induced TAM reprogramming and suppressed tumour growth in preclinical mouse syngeneic and human xenograft tumour models. Surprisingly, combining MAOI and anti-PD-1 treatments resulted in synergistic tumour suppression. In addition, clinical data correlation studies associated high intratumoural MAOA expression with poor patient survival in a broad range of cancers. We further demonstrated that MAO-A promotes TAM immunosuppressive polarization via upregulating oxidative stress. Together, these data identify MAO-A as a critical regulator of TAMs and provide strong evidence for repurposing MAOIs for TAM reprogramming to improve cancer immunotherapies.

The invention disclosed herein has a number of embodiments. Embodiments of the invention include compositions of matter comprising a chemotherapeutic agent: a monoamine oxidase A inhibitor: and a pharmaceutically acceptable carrier. Typically in these embodiments, a monoamine oxidase A inhibitor is present in the composition in such that amounts of monoamine oxidase A inhibitor available for tumor-associated macrophages in an individual administered the composition are sufficient to modulate the phenotype of the tumor-associated macrophages (e.g. wherein modulation of the phenotype comprises decreased levels of intracellular reactive oxygen species: enhanced tumor immunoreactivity: increased expression of CD69, CD86 or MHC class II I-ab: or decreased expression of CD206, or the like).

In certain embodiments of the invention, a monoamine oxidase A inhibitor in the composition comprises at least one of: phenelzine; moclobemide; clorgyline; pirlindole; isocarboxazid; tranylcypromide; iproniazid; caroxazone; befloxatone; brofaromine; cimoxatone; eprobemide; esuprone; metraindol; or toloxatone. Optionally, the monoamine oxidase A inhibitor is disposed within a nanoparticle: for example, a nanoparticle comprising a lipid or the like. The compositions of the invention can include a variety of different chemotherapeutic agents. Optionally for example, a composition of the invention includes at least one immune checkpoint inhibitor chemotherapeutic agent selected to affect CTLA-4 or a PD-1/PD-L1 blockade. In certain embodiments, the checkpoint inhibitor comprises a CTLA-4 blocking antibody, an anti-PD-1 blocking antibody and/or an anti-PD-L1 blocking antibody. In other embodiments, the chemotherapeutic agent comprises carboplatin, cisplatin, paclitaxel, doxorubicin, docetaxel, cyclophosphamide, etoposide, fluorouracil, gemcitabine, methotrexate, erlotinib, imatinib mesylate, irinotecan, sorafenib, sunitinib, topotecan, vincristine, vinblastine, or the like.

Another embodiment of the invention is a method of modulating a phenotype of a tumor-associated macrophage comprising introducing a monoamine oxidase A inhibitor in the environment in which the tumor-associated macrophage is disposed: wherein amounts of the monoamine oxidase A inhibitor introduced into the environment are selected to be sufficient to modulate the phenotype of the tumor-associated macrophage (e.g. wherein modulation of the phenotype comprises decreased levels of intracellular reactive oxygen species: enhanced tumor immunoreactivity: increased expression of CD69, CD86 or MHC class II I-ab: or decreased expression of CD206, or the like). Typically, in these methods, the tumor-associated macrophage is disposed in an individual diagnosed with cancer (e.g. a lymphoma or a skin, breast, ovarian, prostate, colorectal or lung cancer): and the individual is undergoing a therapeutic regimen comprising the administration of a chemotherapeutic agent. Optionally, the monoamine oxidase A inhibitor comprises at least one of phenelzine; moclobemide; clorgyline; pirlindole; isocarboxazid; tranylcypromide; iproniazid; caroxazone; befloxatone; brofaromine; cimoxatone; eprobemide; esuprone; metraindol; or toloxatone, for example one of these compounds disposed within a nanoparticle. These methods of the invention can introduce a monoamine oxidase A inhibitor into an environment in which tumor-associated macrophages are disposed in combination with a variety of different chemotherapeutic agents such as antibodies. Optionally for example, a method of the invention introduces at least one immune checkpoint inhibitor chemotherapeutic agent selected to affect CTLA-4 or a PD-1/PD-L1 blockade. In certain embodiments, the checkpoint inhibitor comprises a CTLA-4 blocking antibody, an anti-PD-1 blocking antibody and/or an anti-PD-L1 blocking antibody. In other embodiments, the chemotherapeutic agent comprises carboplatin, cisplatin, paclitaxel, doxorubicin, docetaxel, cyclophosphamide, etoposide, fluorouracil, gemcitabine, methotrexate, erlotinib, imatinib mesylate, irinotecan, sorafenib, sunitinib, topotecan, vincristine, vinblastine, or the like.

Yet another embodiment of the invention is a method of treating a cancer in an individual comprising administering to the individual a monoamine oxidase A inhibitor: wherein amounts of the monoamine oxidase A inhibitor administered to the individual are selected to be sufficient to modulate the phenotype of tumor-associated macrophages in the individual (e.g. wherein modulation of the phenotype comprises decreased levels of intracellular reactive oxygen species: enhanced tumor immunoreactivity: increased expression of CD69, CD86 or MHC class II I-ab: or decreased expression of CD206). Optionally, the monoamine oxidase A inhibitor comprises at least one of phenelzine: moclobemide: clorgyline: pirlindole: isocarboxazid: tranylcypromide: iproniazid: caroxazone: befloxatone: brofaromine: cimoxatone: eprobemide: esuprone: metraindol: or toloxatone, for example one of these compounds disposed within a nanoparticle. In certain embodiments, the individual is undergoing a therapeutic regimen comprising the administration of at least one chemotherapeutic agent. Some embodiments of the invention include methods of administering monoamine oxidase A inhibitor to the individual in combination with a chemotherapeutic agent. Optionally for example, a method of the invention includes administering a monoamine oxidase A inhibitor to the individual in combination with at least one immune checkpoint inhibitor chemotherapeutic agent selected to affect CTLA-4 or a PD-1/PD-L1 blockade. In certain embodiments, the checkpoint inhibitor comprises a CTLA-4 blocking antibody, an anti-PD-1 blocking antibody and/or an anti-PD-L1 blocking antibody. In other embodiments of the invention, the chemotherapeutic agent comprises carboplatin, cisplatin, paclitaxel, doxorubicin, docetaxel, cyclophosphamide, etoposide, fluorouracil, gemcitabine, methotrexate, erlotinib, imatinib mesylate, irinotecan, sorafenib, sunitinib, topotecan, vincristine, vinblastine, or the like.

Other objects, features and advantages of the present invention will become apparent to those skilled in the art from the following detailed description. It is to be understood, however, that the detailed description and specific examples, while indicating some embodiments of the present invention, are given by way of illustration and not limitation. Many changes and modifications within the scope of the present invention may be made without departing from the spirit thereof, and the invention includes all such modifications.

### BRIEF DESCRIPTION OF THE DRAWINGS

FIG. 1: MAO-A-deficient mice show reduced tumour growth associated with altered TAM polarization. a, QPCR analyses of Maoa mRNA expression in TAMs isolated from wildtype mice bearing B16-OVA tumours. Monocytes (Mo) isolated from tumour-free and tumour-bearing mice were included as controls. N=4. b-d, Growth of B16-OVA tumours in Maoa WT and Maoa KO mice. (b) Experimental design. (c) Tumour growth. (d) Tumour volume at day 18. N=8-9. e-j, Phenotype of TAMs isolated from Maoa WT and Maoa KO mice bearing B16-OVA tumours, at day 18 post tumour challenge. (e-h) FACS analyses of CD206 (e), CD69 (f), CD86 (g), and I-Ab (h) expression on TAMs (n=8-9). MFI, mean fluorescence intensity. (i, j) QPCR analyses of immunosuppressive (Mrc1, Chi3/3, and Arg1: i) and immunostimulatory (I16, Cc12, and Tnf: j) signature gene mRNA expression in TAMs (n=4). k-n, scRNAseq analyses of tumour-infiltrating immune cells (TIIs) isolated from Maoa WT and Maoa KO mice bearing B16-OVA tumours, at day 14 post tumour challenge. (k) Uniform Manifold Approximation and Projection (UMAP) of single TIIs showing the formation of 6 cell clusters (TAM/Mono, T cell, NK cell, B cell, DC, and pDC) from total CD45.2⁺ TIIs and 5 cell clusters (TAM1, TAM2, Mono1, Mono2, and Mono3) from the TAM/Mono subpopulation. Each dot represents one single cell and is colored according to cell types. Mono, monocyte: NK, natural killer cell: DC, dendritic cell: pDC, plasmacytoid dendritic cell. (l) UMAP of the TAM subpopulation, showing the formation of two clusters (TAMI: Mrc1^{low}Cd86^{high}; and TAM2: Mrc1^{high}86^{low}). Each dot represents one single cell and is colored according to cell clusters. Ratios of TAM1:TAM2 are presented. (m, n) Violin plots showing the expression distribution of immunosuppressive (Mrc1 and Chi313: m) and immunostimulatory (Cc12, Cc17, Cd86, H2-Aa, and H2-Ah1: n) signature genes in single TAMs. Each dot represents an individual cell. Representative of 1 (k-n), 3 (a), and 5 (b-j) experiments. All data are presented as the mean±SEM. *P<0.05, **P<0.01, and ***P<0.001, by 1-way ANOVA (a) or by Student's t test (c-j). P values of violin plots were determined by Wilcoxon rank sum test (m, n).

FIG. 2: MAO-A directly regulates TAM polarization and influences TAM-associated antitumour T cell reactivity. a-f, Studying B16-OVA tumour growth and TAM phenotype in BoyJ (CD45.1) wildtype mice reconstituted with bone marrow cells isolated from either Maoa WT or Maoa KO donor mice (denoted as WT or KO experimental mice, respectively). (a) Experimental design. (b) Tumour growth. (c) Tumour volume at day 24. (d-f) FACS analyses of CD206 (d), CD69 (e), and CD86 (f) expression on TAMs at day 24. N=8-9. g-m, Studying B16-OVA tumour growth and antitumour T cell reactivity in a Tumour-TAM Co-Inoculation in vivo experiment. BoyJ wildtype mice received s.c. inoculation of B16-OVA tumour cells mixed with either Maoa WT or Maoa KO BMDMs (denoted as WT or KO experimental mice, respectively). BMDM, bone marrow-derived macrophage. (g) Experimental design. (h) Tumour growth (n=9-10). (i) Tumour volume at day 18 (n=9-10). (j-l) FACS analyses of CD206 (j), CD69 (k), and CD86 (l) expression on CD45.2* TAMs at day 6 (n=8). (m) FACS analyses of intracellular Granzyme B production in tumour-infiltrating CD45.1*CD8⁺ T cells at day 18 (n=9-10). Representative of 3 experiments. All data are presented as the mean±SEM. *P<0.05, **P<0.01, and ***P<0.001, by Student's t test.

FIG. 3: MAO-A promotes macrophage immunosuppressive polarization. a-g, Studying the in vitro differentiation and IL-4/IL-13-induced polarization of Maoa WT (WT) and Maoa KO (KO) BMDMs. (a) Experimental design. (b,c) QPCR analyses of Maoa mRNA expression over the 6-day BMDM differentiation culture (b) and IL-4/IL-13-induced polarization (c) (n=6). (d) Western blot analyses of MAOA protein expression in the indicated BMDMs. (e) FACS analyses of CD206 expression on the indicated BMDMs (n=4). (f,g) QPCR analyses of Chi3/3 (f) and Arg1 (g) mRNA expression in the indicated BMDMs (n=4). NC, no cytokine control BMDMs: IL-4/IL-13, IL-4 and IL-13-polarized BMDMs. h-k, Studying the T cell suppression function of Maoa WT (WT) and Maoa KO (KO) IL-4/IL-13-polarized BMDMs in an in vitro macrophage/T cell co-culture assay (n=3). (h) Experimental design. (i) FACS quantification of CD8⁺ T cells (identified as TCRβ⁺CD4⁻CD8⁺ cells). (j,k) FACS analyses of CD25 (j) and CD62L (k) expression on CD8⁺ T cells. i-p, Studying the IL-4/IL-13-induced polarization of Maoa KO BMDMs with MAO-A overexpression (n=3). In vitro-cultured Maoa KO BMDMs were transduced with either a MIG-Maoa retrovector or a MIG mock retrovector, polarized with IL-4/IL-13, followed by FACS sorting of GFP⁺ Maoa KO BMDMs for further analyses. (l) Schematics of the MIG and MIG-Maoa retrovectors. (m) FACS analyses of prior-to-sorting Maoa KO BMDMS, showing retrovector transduction efficiency (measured as % GFP⁺ cells). (n-p) QPCR analyses of sorted GFP⁺ Maoa KO BMDMs, showing the mRNA expression of Maoa (n), Chi313 (o), and Arg1 (p). Representative of 3 (h-k, l-p) and 4 (a-g) experiments. All data are presented as the mean±SEM. ns, not significant, **P<0.01, and ***P<0.001, by 1-way ANOVA (b), 2-way ANOVA (e-g, i-k), or Student's t test (c, n-p).

FIG. 4: MAO-A promotes macrophage immunosuppressive polarization via ROS upregulation. a, Schematics showing the enzymatic activity of MAO-A in a TAM. MAO-A breaks down monoamines and generates hydrogen peroxide (H₂O₂) as a byproduct, thereby increasing reactive oxygen species (ROS) levels in a TAM. b,c, Studying the in vivo ROS levels in TAMs isolated from Mao WT and Mao KO mice bearing B16-OVA tumours (n=4). (b) Experimental design. (c) FACS analyses of ROS levels in Maoa WT and Maoa KO TAMs at day 18. TAMs were gated as the CD45.2⁺CD11b⁺Ly6G⁻Ly6C^{-/low}F4/80⁺ cells of total tumour-infiltrating immune cells (TIIs). d, FACS analyses of ROS levels in in vitro-cultured Maoa WT and Maoa KO BMDMs, without or without IL-4/IL-13 polarization for 24 hours (n=4). NC, no cytokine-treated control BMDMs: IL-4/IL-13, IL-4/IL-13-polarized BMDMs. e-g, Studying the phenotype of IL-4/IL-13-polarized Maoa WT and Maoa KO BMDMs with or without H₂O₂ treatment (n=3). BMDMs were treated with H₂O₂ for 30 minutes prior to IL-4/IL-13 polarization for 24 hours. (e) FACS analyses of CD206 expression on BMDMs. (f,g) QPCR analyses of Chi313 (f) and Arg1 (g) mRNA expression in BMDMs. h-j, Studying the phenotype of IL-4/IL-13-polarized Maoa WT and Maoa KO BMDMs with or without tyramine supplement (n=3). BMDMs were treated with tyramine for 30 minutes prior to IL-4/IL-13 polarization for 24 hours. (h) FACS analyses of ROS levels in BMDMs. (i,j) QPCR analyses of Chi3/3 (i) and Arg1 (j) mRNA expression in BMDMs. k,l, Studying the in vivo Stat6 signaling in TAMs isolated from Mao WT and Mao KO mice bearing B16-OVA tumours (combined from 5 mice per group). (k) Experimental design. (l) Western blot analyses of Stat6 phosphorylation in TAMs at day 18. TAMs were FACS sorted as the DAPI⁻CD45.2⁺CD11b⁺Ly6G⁻Ly6C^{-/low}F4/80⁺ cells from total TIIs. m, Western blot analyses of JAK-Stat6 signaling in in vitro-cultured Maoa WT and Maoa KO BMDMs, with or without IL-4/IL-13 polarization and H₂O₂ treatment. BMDMs were treated with H₂O₂ for 30 minutes prior to IL-4/IL-13 stimulation for another 30 minutes. Representative of 3 experiments. All data are presented as the mean±SEM. ns, not significant, *P<0.05, **P<0.01, and ***P<0.001, by 2-way ANOVA (d-j) or by Student's t test (c).

FIG. 5: MAO-A blockade for cancer immunotherapy-syngeneic mouse tumour model studies. a-e, Studying the effect of MAOI treatment on IL-4/IL-13-induced BMDM polarization in vitro (n=4). (a) Experimental design. Wildtype BMDMs were stimulated with IL-4/IL-13 with or without MAOI treatment. MAOIs (monoamine oxidase inhibitors) studied were phenelzine (Phe: 20 μM), clorgyline (Clo: 20 μM), moclobemide (Moc: 200 μM), and pirlindole (Pir: 20 μM). NT, no MAOI treatment. (b) FACS analyses of ROS levels in BMDMs. (c) FACS analyses of CD206 expression on BMDMs. (d,e) QPCR analyses of Chi313 (d) and Arg1 (e) mRNA expression in BMDMs. f-j, Studying the TAM-related cancer immunotherapy potential of MAOI treatment in a B16-OVA melanoma syngeneic mouse tumour model (n=7-8). (f) Experimental design. B6 wildtype mice were treated with clodronate liposomes (Clod) to serve as TAM-depleted experimental mice, or treated with vehicle liposomes (Veh) to serve as TAM-intact control mice. Phe, phenelzine treatment: NT, no phenelzine treatment. (g) Tumour growth. (h) Tumour volume at day 18. (i) FACS analyses of CD206 expression on TAMs of TAM-intact experimental mice. (j) FACS analyses of intracellular Granzyme B production in tumour-infiltrating CD8⁺ T cells of all experimental mice. k-o, Studying the cancer therapy potential of MAOI treatment in combination with anti-PD-1 treatment in the B16-OVA melanoma and MC38 colon cancer syngeneic mouse tumour models (n=5). (k) Experimental design. Tumour-bearing mice were treated with anti-PD-1 antibody (aPD-1) or isotype control (Iso), together with or without phenelzine (Phe) treatment. NT, no Phe treatment. (l) B16-OVA tumour growth. (m) B16-OVA tumour volume at day 18. (n) MC38 tumour growth. (o) MC38 tumour volume at day 27. Representative of 3 experiments. All data are presented as the mean±SEM. ns, not significant, *P<0.05, **P<0.01, and ***P<0.001, by I-way ANOVA (b-e, h, j, m, o) or by Student's t test (i).

FIG. 6: MAO-A blockade for cancer immunotherapy-human TAM and clinical data correlation studies. a, Studying the MAOA gene expression in human M1- and M2-like macrophages. A transcriptome data set (GSE35449) was analyzed using the prioritization function of a Tumour Immune Dysfunction and Exclusion (TIDE) computational method. Heatmaps are presented, showing the M2-like/M1-like mRNA fold change of MAOA gene as well as a selected group of immunosuppressive, immunostimulatory, and immune checkpoints signature genes. b-d, Studying the MAO-A expression in in vitro differentiated and IL-4/IL-13-polarized human monocyte-derived macrophages (MDMs: n=4). MDMs were generated by culturing healthy donor peripheral blood monocytes over 6 days, followed by stimulation with IL-4 and IL-13 for another 2 days. NC, no cytokine stimulation. (b,c) QPCR analyses of MAOA mRNA expression in MDMs over the 6-day MDM differentiation culture (b) and post the IL-4/IL-13-induced polarization (c). (d) Western blot analyses of MAO-A protein expression in IL-4/IL-13-polarized MDMs. e-g, Studying the in vitro polarization of human MDMs (n=3). MDMs were stimulated with IL-4/IL-13 for 2 days, in the presence or absence of phenelzine (Phe, 20 μM) treatment. NC, no cytokine stimulation: NT, no phenelzine treatment. (e) FACS analyses of CD206 expression on MDMs. (f,g) QPCR analyses ALOX15 (f) and (CD200R1 (g) mRNA expression in MDMs. h-j, Studying the in vivo polarization of human macrophages in a human Tumour-TAM Co-Inoculation xenograft mouse model (n=4). (h) Experimental design. FACS-sorted healthy donor peripheral blood monocytes were mixed with human A375 melanoma cells and s.c. injected into NSG mice to form solid tumours, with or without phenelzine treatment (Phe or NT). (i,j) FACS analyses of CD206 (i) and CD273 (j) expression on TAMs (gated as hCD45⁺hCD11b⁺hCD14⁺ cells of total TIIs) isolated from experimental mice at day 10. k-m, Studying the in vitro efficacy of phenelzine in reprogramming human TAMs and enhancing human T cell antitumour reactivity (n=6). (k) Schematics showing an in vitro 3D human tumour/TAM/T cell organoid culture. A375-A2-ESO, human A375 melanoma cell line engineered to express an NY-ESO-1 tumour antigen as well as its matching HLA-A2 molecule: ESO-T, human peripheral blood CD8 T cells engineered to express an NY-ESO-1-specific TCR: Polarized TAM, human MDMs polarized in vitro with IL-4/IL-13 in the presence or absence of phenelzine treatment (denoted as TAM Phe or TAM NT, respectively). Cells were mixed and cultured as organoids for two days before analysis. (l,m) FACS quantification of live tumour cells (gated as hCD45⁻ cells) and ESO-T cells (gated as hCD45⁺hCD8⁺ESO-TCR⁺ cells). n, QPCR analyses of MAOA mRNA expression in human TAMs isolated from ovarian cancer patient tumour samples (n=4). Monocytes isolated from random healthy donor peripheral blood were included as controls (n=10). Mo, monocyte. o-r, Clinical data correlation studies. The correlation function of a TIDE computational method was utilized. The association between the intratumoural MAOA gene expression levels and overall survival (OS) of cancer patients was computed through the two-sided Wald test in the Cox-PH regression. For each patient cohort, tumour samples were divided into the MAOA-high (samples with MAOA expression one standard deviation above the average) and MAOA-low (remaining samples) groups, followed by analysing the OS of each group. TIDE analyses of an ovarian cancer patient cohort (GSE26712, n=182: o), a lymphoma patient cohort (GSE10846, n=388: p), a breast cancers patient cohort (GSE9893, n=148: q), and a melanoma patient cohort with anti-PD-1 therapy (PRJEB23709, n=41: r). Representative of 1 (n), 2 (b-d, h-j) and 3 (e-g, k-m) experiments. All data are presented as the mean±SEM. ns, not significant, *P<0.05, **P<0.01, and ***P<0.001, by 1-way ANOVA (l, m), 2-way ANOVA (e-g), or by Student's t test (i, j, n). For Kaplan-Meier plots, the P value was calculated by two-sided Wald test in a Cox-PH regression (o-r).

FIG. 7: MAO-A-deficient mice show reduced tumour growth associated with altered TAM polarization. a, Western blot analyses of MAO-A protein expression in spleen (SP) and bone marrow (BM) cells harvested from Maoa WT (WT) and Maoa KO (KO) mice. b-d, Phenotypes of TAMs and tumour-infiltrating CD8⁺ T cells isolated from Maoa WT and Maoa KO mice bearing B16-OVA tumours, at day 18 post tumour challenge (n=8-9). (b) FACS gating strategy to identify TAMs (gated as CD45.2⁺CD11b⁺Ly6G⁻Ly6C^{-/low}F4/80⁺ cells) from total tumour-infiltrating immune cells (TIIs). (c) FACS quantification of TAMs. (d) FACS analyses of intracellular Granzyme B production in tumour-infiltrating CD8⁺ T cells (gated as CD45.2⁺TCRβ⁺CD8⁺ cells from total TIIs). e-g, scRNAseq analyses of TIIs isolated from Maoa WT or Maoa KO mice bearing B16-OVA tumours, at day 14 post tumour challenge. Uniform Manifold Approximation and Projection (UMAP) plots are presented. Each dot represents one single cell and is colored according to the expression level of an indicated gene. (e) UMAP of single TIIs, showing the expression patterns of 7 marker genes (Cd3d, Gzma, Itgam, Cd79a, Siglech, Cd209a, and Flt3) used to define 6 cell clusters (T, B, NK, DC, pDC, and TAM/Mono). (f) UMAP of single cells of the TAM/Mono subpopulation, showing the expression patterns of 3 marker genes (Ly6c2, C1qc, and Itgam) used to define 5 cell clusters (TAM1, TAM2, Mono1, Mono2, and Mono3). (g) UMAP of single cells of the TAMI and TAM2 subpopulations, showing the expression patterns of a pair of immunosuppressive and immunostimulatory signature genes (Mrc1 and Cd86, respectively). Representative of 1 (e-g), 2 (a), and 5 (b-d) experiments. All data are presented as the mean±SEM. ns, not significant, **P<0.01, by Student's t test.

FIG. 8: MAO-A directly regulates TAM polarization and influences TAM-associated antitumour T cell reactivity. BoyJ (CD45.1) wildtype mice reconstituted with bone marrow cells harvested from Maoa WT or Maoa KO donor mice (denoted as WT or KO mice, respectively) were inoculated with B16-OVA tumour cells. At day 18 post tumour challenge, TIIs were isolated from the experimental mice for FACS analysis. N=7-9. a, FACS analyses of I-Ab expression on TAMs (gated as CD45.2⁺CD11b⁺Ly6G^{-/low}F4/80⁺ cells of total TIIs). b, FACS analyses of Granzyme B intracellular production in tumour-infiltrating CD8⁺ T cells (gated as CD45.2+TCRβ⁺CD8⁺ cells of total TIIs). Representative of 3 experiments. All data are presented as the mean±SEM. **P<0.01, by Student's t test.

FIG. 9: MAO-A promotes macrophage immunosuppressive polarization. a, Studying the T cell suppression function of Maoa WT (WT) and Maoa KO (KO) IL-4/IL-13-polarized BMDMs in an in vitro macrophage/T cell co-culture assay (n=3). Polarized BMDMs were mixed with 1×10⁶ splenocytes harvested from B6 wildtype mice at 0:1, 1:2, 1:4, or 1:8 ratios. FACS quantifications of CD44 expression on CD8⁺ T cells (identified as CD11b⁻TCRβ⁺CD8⁺ cells) are presented. b, Schematics showing the experimental design to overexpress MAO-A in Maoa KO BMDMs. Representative of 3 (a) experiments. All data are presented as the mean±SEM. ns, not significant, **P<0.01, ***P<0.001, by 2-way ANOVA (A).

FIG. 10: MAO-A promotes macrophage immunosuppressive polarization via ROS upregulation. Moon WT and Moon KO BMDMs (denoted as WT and KO, respectively) were treated with H₂O₂ for 30 minutes followed by IL-4/IL-13 stimulation for another 30 minutes. BMDMs were then collected for FACS analysis. N=4. a, FACS plots showing ROS levels in the indicated BMDMs. b, Quantification of A. Representative of 2 experiments. All data are presented as the mean±SEM. ns, not significant, ***P<0.001, by 2-way ANOVA (b).

FIG. 11: MAO-A blockade for cancer immunotherapy-syngeneic mouse tumour model studies. a, Efficient depletion of TAMs in B6 wildtype mice bearing B16-OVA tumours through clodronate liposome treatment (Clod). Tumour-bearing mice treated with vehicle liposomes (Veh) were included as a control. The experimental design is shown in main FIG. 5F. FACS quantifications of TAMs (gated as CD45.2⁺CD11b⁺Ly6G⁻Ly6C^{-/low}F4/80⁺ cells of total TIIs) are presented (n=7-8). b-f, Studying B16-OVA and MC38 tumour growth in B6 wildtype mice with or without phenelzine treatment (Phe or NT). (b) Experimental design. (c) B16-OVA tumour growth (n=10). (d) B16-OVA tumour volume at day 18. (e) MC38 tumour growth (n=6). (f) MC38 tumour volume at day 27. g-k, Studying B16-OVA and MC38 tumour growth in NSG mice with or without phenelzine treatment (Phe or NT: n=5). (g) Experimental design. (h) B16-OVA tumour growth. (i) B16-OVA tumour volume at day 18. (j) MC38 tumour growth. (k) MC38 tumour volume at day 21. Representative of 2 (a-f) and 4 (g-k) experiments. All data are presented as the mean±SEM. ns, not significant, **P<0.01, ***P<0.001, by Student's t test (a, d, f, i, k).

FIG. 12: MAO-A blockade for cancer immunotherapy-human TAM and clinical data correlation studies. a, Studying the IL-4/IL-13-induced in vitro polarization of human monocyte-derived macrophages (MDMs) in the presence or absence of phenelzine (Phe) treatment. NC, no cytokine treatment: NT, no Phe treatment. FACS analyses of CD273 expression on MDMs are presented (n=3). b, FACS gating strategy to identify human TAMs (gated as hCD45⁺hCD11b⁺hCD14⁺ cells of total TIIs) in a human Tumour-TAM Co-Inoculation xenograft mouse model. The experimental design is shown in main FIG. 6H. c,d, Generation of the A375-A2-ESO human melanoma cell line. (c) Experimental design. The A375-A2-ESO cell line was generated by stably co-transducing the parental A375 human melanoma cell line with a Lenti/HLA-A2 lentivector encoding the human HLA-A2 molecule and a Lenti/NY-ESO-1 lentivector encoding the human NY-ESO-1 tumour antigen. (d) FACS plots showing the detection of HLA-A2 molecule and NY-ESO-I tumour antigen (indicated by RFP) on A375-A2-ESO cells. The parental A375 cells were included as a staining control. e,f, Generation of the ESO-T cells. (e) Experimental design. Human peripheral blood mononuclear cells (hPBMCs) from healthy donors were stimulated in vitro with anti-CD3/CD28 and IL-2 to expand human CD8⁺ T cells, followed by transduction with a Retro/ESO-TCR retrovector encoding an HLA-A2-restricted NY-ESO-1 specific TCR (clone 3A1). The resulting human CD8⁺ T cells, denoted as the ESO-T cells, can specifically target the A375-A2-ESO human melanoma cells. (f) FACS plots showing the transduction efficiency of the engineered human CD8⁺ ESO-T cells. Human CD8⁺ T cells that received mock transduction were included as a staining control (denoted as Mock-T). g, Studying the in vitro efficacy of phenelzine in reprogramming human TAMs and enhancing human T cell antitumour reactivity in an in vitro 3D human tumour/TAM/T cell organoid culture. The experimental design is shown in main FIG. 6K. FACS plots showing the surface expression of CD25 and CD62L on ESO-T cells (n=6). h, FACS sorting of human TAMs from primary ovarian cancer patient tumour samples. Tumour-infiltrating immune cells were isolated from fresh ovarian cancer patient tumour samples and then were subjected to FACS sorting to isolate TAMs (identified as DAPI⁻hCD45⁺hCD11b⁺hTCRαβ⁻hCD14⁺ cells). Representative FACS plots are presented (n=4). i, FACS sorting of primary human monocytes from random healthy donor blood samples. PBMCs were subjected to FACS sorting to isolate monocytes (identified as DAPI⁻hCD45⁺hCD11b⁺hTCRαβ⁻hCD14⁺ cells). Representative FACS plots are presented (n=10). Representative of 3 (a-g) and 4 (h-i) experiments. All data are presented as the mean±SEM. **P<0.01, ***P<0.001, by 2-way ANOVA (a, g).

FIG. 13: The “intratumoural MAO-A-ROS axis” model. Schematics showing the “intratumoural MAO-A-ROS axis” model. (Left Panel) Function of MAO-A in the brain. Neurons express MAO-A (as well as its isoenzyme MAO-B) that degrades monoamine neurotransmitters (e.g., dopamine, noradrenaline, and serotonin), thereby regulating neuron signal transmission. Meanwhile, the enzymatic activity of MAO-A generates hydrogen peroxide as a byproduct and thereby upregulating ROS levels (hence, oxidative stress) in neurons. Excessive oxidative stress induces the destruction of neuron cellular components and ultimately leading to neurodegeneration and neuron death. Small molecule monoamine oxidase inhibitors (MAOIs) have been developed and clinically utilized for treating neuropsychiatric disorders, such as depression, and neurodegeneration diseases, such as Parkinson's disease. (Right Panel) Function of MAO-A in a tumour. Analogous to neurons in the brain, TAMs in the tumour microenvironment also express MAO-A, that controls TAM intracellular ROS levels by hydrogen peroxide production, thereby regulating TAM immunosuppressive polarization and subsequently CD8⁺ T cell antitumour reactivity. Established MAOI antidepressants can potentially be repurposed for improving cancer immunotherapy, through targeting the “MAO-A-ROS axis” of TAM polarization in tumours. Notably, unlike neurons that co-express MAO-A and MAO-B, TAMs in particular the immunosuppressive TAMs predominantly express MAO-A.

FIG. 14: MAOA:MAOB gene expression profile in human macrophages. Comparing MAOA:MAOB gene expression ratio in human M0-, M1- and M2-like macrophages by analyzing a transcriptome data set (GSE35449). Each dot represents one single sample (n=7). All data are presented as the mean±SEM. ns, not significant, **P<0.01, ***P<0.001, by 1-way ANOVA.

FIG. 15: Delivery of pheneizine using cMLV. (A) Schematics of cMLV. (B-C). Study the cancer therapy potential of cMLV-formulated phenelzine (cMLV-Phe, 30 mg/kg) in a B16-OVA mouse melanoma model. Free phenelzine (Free-Phe, 30 mg/kg) was included as a control. (B) Experimental design. (C) Tumor growth (n=5). Data are presented as the mean±SEM. ns, not significant, ***P<0.001, ****P<0.0001, by one-way ANOVA.

FIG. 16. Behavioral study of B16-OVA tumor-bearing mice treated with either free or cMLV-formulated pheneizine (Free-Phe or cMLV-Phe; i.v., q3d). N=5-6. (A) Percentage of animals showing medium to strong aggression. (B) Quantification of aggression bouts per trial across different conditions. (C) Quantification of latency to the onset of aggression in each trial across different conditions. (D) Quantification of total time the animals engage in aggressive behavior in each trial across different conditions. (E) Representative raster plots showing aggression. (F) Phenelzine (Phe) measurements in the brain (n=3). Data are presented as the mean±SEM. ***P<0.001, by one-way ANOVA.

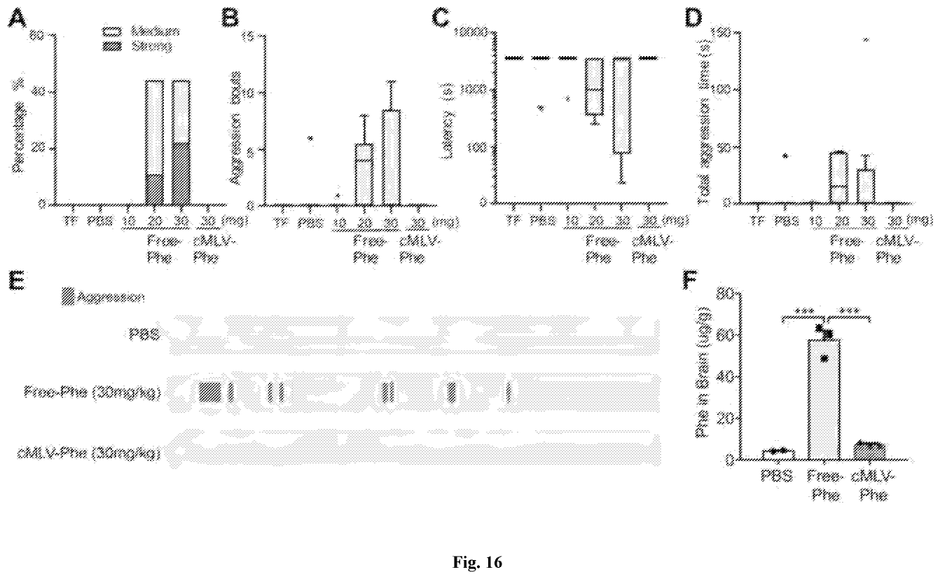

### DETAILED DESCRIPTION OF THE INVENTION

In the description of embodiments, reference may be made to the accompanying figures which form a part hereof, and in which is shown by way of illustration a specific embodiment in which the invention may be practiced. It is to be understood that other embodiments may be utilized, and structural changes may be made without departing from the scope of the present invention. Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. Many of the aspects of the techniques and procedures described or referenced herein are well understood and commonly employed by those skilled in the art. The following text discusses various embodiments of the invention.

Monoamine oxidase A (MAO-A) is an outer mitochondrial membrane-bound enzyme encoded by the X-linked MAOA gene. MAO-A is best known for its function in the brain, where it is involved in the degradation of a variety of monoamine neurotransmitters, including serotonin, dopamine, epinephrine, and norepinephrine. Through regulating the availability of serotonin, MAOA modulate neuronal activities thereby influencing mood and behavior in humans^{43, 44, 45, 46, 47}. Through regulating the availability of dopamine and the abundance of dopamine breakdown byproduct hydrogen peroxide (H₂O₂: hence oxidative stress), MAO-A is involved in multiple neurodegenerative diseases, including Parkinson's disease (PD)^{48, 49}. FDA-approved small-molecule MAO inhibitors (MAOIs) are currently available for the treatment of neurological disorders, including depression and PD^{47, 49, 50, 51, 52, 53, 54, 55}. However, the functions of MAO-A outside of the brain are largely unknown. In this study, we investigated the role of MAO-A in regulating TAM polarization and evaluated the possibility of repurposing MAOIs for reprogramming TAMs and improving cancer immunotherapy, using knockout and transgenic mice, preclinical mouse syngeneic and human xenograft tumour models, as well as human TAM and clinical data correlation studies.

The disclosure provided herein identifies MAO-A as an immune checkpoint and the use of MAOI antidepressants to modulate TAM phenotypes for cancer immunotherapy. The invention disclosed herein has a number of embodiments. Embodiments of the invention include compositions of matter comprising a chemotherapeutic agent: a monoamine oxidase A inhibitor: and a pharmaceutically acceptable carrier. Typically in these embodiments, a monoamine oxidase A inhibitor is present in the composition in such that amounts of monoamine oxidase A inhibitor available for tumor-associated macrophages in an individual administered the composition are sufficient to modulate the phenotype of the tumor-associated macrophages (e.g. wherein modulation of the phenotype comprises decreased levels of intracellular reactive oxygen species: enhanced tumor immunoreactivity: increased expression of CD69, CD86 or MHC class II I-ab: or decreased expression of CD206, or the like).

In certain embodiments of the invention, a monoamine oxidase A inhibitor in the composition comprises at least one of: phenelzine; moclobemide; clorgyline; pirlindole; isocarboxazid; tranylcypromide; iproniazid; caroxazone; befloxatone; brofaromine; cimoxatone; eprobemide; esuprone; metraindol; or toloxatone. Optionally, the monoamine oxidase A inhibitor is disposed within a nanoparticle; for example, a nanoparticle comprising a lipid or the like. In particular, embodiments of the invention can utilize such nanocarriers to address the short circulatory half-life of free MAOI; limited cancer targeting/penetration; and toxicity of MAOI in CNS. Illustrative nanocarriers include lipid-coated mesoporous silica nanoparticles (“silicasomes”) as well as liposome platforms. In certain embodiments, the nanocarrier is designed to have a size, a charge, one or more surface coatings (e.g., PEG, copolymers), one or more targeting ligands (e.g., peptides) and the like: an optionally the inclusion of imaging agents and the like, with a view to obtaining colloidal stability, low opsonization, long circulatory t1/2, and effective biodistribution post intravenous (IV) injection.

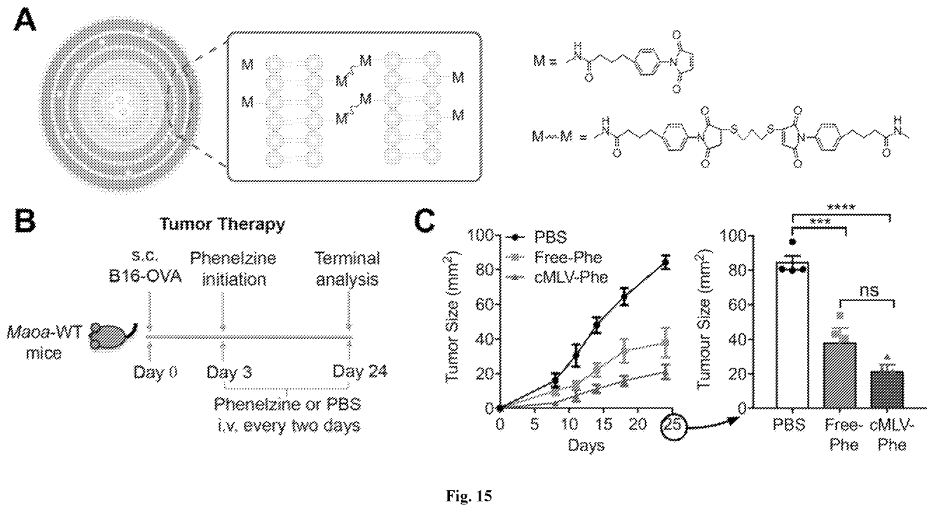

One such nanocarrier embodiment comprises the irreversible, non-selective MAOI phenelzine because its chemical properties (water solubility of 11.1 mg/mL, LogP 1.2 and pKa 5.5). Other possible MAOIs that are suitable for loading include isocarboxazid and tranylcypromine. Liposomes can be synthesized using lipid biofilm, rehydration, sonication and extrusion (e.g., using membrane of 100 nm pore size) protocols. One can, for example, use a lipid bilayer that exhibits an DSPC/Cholesterol/DSPE-PEG2000 at molar ratio 3:2:0.15. For silicasome embodiments, a bare MSNP core can be constructed using a templating agent and silica precursors to make 80˜90 nm particles. The particles can be produced in big batch sizes (e.g., ˜5 g/batch) and stably stored for 18˜24 months, allowing aliquots to be removed at different project stages for carrier development. Phenelzine can be remotely imported using different trapping agents, such as triethylammmonium sucrose octasulfate, (NH4)2SO4 or citric acid. Lipid coatings can be introduced using ethanol injection method with controlled sonication power. Data showing a working embodiment of the invention comprising a crosslinked multilamellar liposome is shown in FIG. 15.

In certain embodiments of the invention, the monoamine oxidase A inhibitor is disposed within a composition comprising a crosslinked multilamellar liposome having an exterior surface and an interior surface, the interior surface defining a central liposomal cavity, the multilamellar liposome including at least a first lipid bilayer and a second lipid bilayer, the first lipid bilayer being covalently bonded to the second lipid bilayer: and the monoamine oxidase A inhibitor disposed within the liposome (see, e.g. FIG. 15). Such liposome compositions are known in the art and discussed, for example, in: U.S. Patent Application Publication No. 20140356414: Joo et al. Biomaterials 34, 3098-3109, doi: 10.1016/j.biomaterials.2013.01.039 (2013); Liu et al., Biomed Res Int 2013, 378380, doi:10.1155/2013/378380 (2013): Liu et al., Mol Pharm 11, 1651-1661, doi:10.1021/mp5000373 (2014): Liu et al., PLoS One 9, el 10611, doi:10.1371/journal.pone.0110611 (2014): Kim, Y. J. et al. Mol Pharm 12, 2811-2822, doi:10.1021/mp500754r (2015): and Zhang, X. et al. RSC Adv 7, 19685-19693, doi:10.1039/c7ra01100h (2017), the contents of which are incorporated herein by reference.

Based on the growing awareness that tumor targeting and/or the activation of tumor transcytosis mechanism may generate more robust access in multiple solid tumors, we can make nanoparticles having targeting agents by introducing peptide conjugation to the LB (e.g., iRGD and tumor targeting Arg-Gly-Asp peptide), using a thiol-maleimide reaction to link the cysteine-modified peptide to DSPE-PEG2000-maleimide. All the MAOI nanocarriers can be thoroughly characterized for physicochemical properties, such as size, morphology (cryoEM), loading capacity, release profile, zeta potential, impurities, and stability in biological fluids before use. The biological activity of nMAOIs can be read out by measuring nMAOI regulation of TAMS.

In certain embodiments of the invention, the monoamine oxidase A inhibitor is present in the composition in specific amounts such as at least 100 mg, or at least 250 mg. or at least 500 mg (e.g., of moclobemide). However, in view of the fact that different people weigh different amounts and may respond differently to a specific amount of a monoamine oxidase A inhibitor, those of skill in this art understand that a more precise way to describe embodiments of the invention is to include a description of what the composition does (e.g. decreases levels of intracellular reactive oxygen species: enhances tumor immunoreactivity: increases expression of CD69, CD86 or MHC class II I-ab: or decreases expression of CD206, or the like), rather than by what the composition is (e.g. 100 mg of a monoamine oxidase A inhibitor). In view of the well-studied pharmacology of monoamine oxidase A inhibitors, the disclosure provided herein along with the known pharmacodynamics of monoamine oxidase A inhibitors (see, e.g., Holford et al: Br J Clin Pharmacol. 1994 May:37(5):433-9 for moclobemide) makes the dosing associated with a desired effect to be routine in the art.

The compositions of the invention can include a variety of different chemotherapeutic agents. Optionally for example, a composition of the invention includes at least one immune checkpoint inhibitor chemotherapeutic agent selected to affect CTLA-4 or a PD-1/PD-L1 blockade. In certain embodiments, the checkpoint inhibitor comprises a CTLA-4 blocking antibody, an anti-PD-1 blocking antibody and/or an anti-PD-L1 blocking antibody. In other embodiments, the chemotherapeutic agent comprises carboplatin, cisplatin, paclitaxel, doxorubicin, docetaxel, cyclophosphamide, etoposide, fluorouracil, gemcitabine, methotrexate, erlotinib, imatinib mesylate, irinotecan, sorafenib, sunitinib, topotecan, vincristine, vinblastine, or the like.

The compositions of the invention comprising monoamine oxidase A inhibitor may be made and then systemically administered in combination with a pharmaceutically acceptable vehicle such as an inert diluent. For oral therapeutic administration, the compounds may be combined with one or more excipients and used in the form of ingestible tablets, buccal tablets, troches, capsules, elixirs, suspensions, syrups, wafers, and the like. For compositions suitable for administration to humans, the term “excipient” is meant to include, but is not limited to, those ingredients described in Remington: The Science and Practice of Pharmacy, Lippincott Williams & Wilkins, 21st ed. (2006) (hereinafter Remington's). Common illustrative excipients include antimicrobial agents and buffering agents.

The compositions of the invention comprising monoamine oxidase A inhibitor may be administered parenterally, such as intravenously or intraperitoneally by infusion or injection. Solutions of the compositions of the invention comprising monoamine oxidase A inhibitor can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations can contain a preservative to prevent the growth of microorganisms.

The pharmaceutical dosage forms suitable for injection or infusion can include sterile aqueous solutions or dispersions or sterile powders comprising compounds which are adapted for the extemporaneous preparation of sterile injectable or infusible solutions or dispersions, optionally encapsulated in liposomes. In all cases, the ultimate dosage form should be sterile, fluid, and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof.

Another embodiment of the invention is a method of modulating a phenotype of a tumor-associated macrophage comprising introducing a monoamine oxidase A inhibitor in the environment in which the tumor-associated macrophage is disposed: wherein amounts of the monoamine oxidase A inhibitor introduced into the environment are selected to be sufficient to modulate the phenotype of the tumor-associated macrophage (e.g. wherein modulation of the phenotype comprises decreased levels of intracellular reactive oxygen species: enhanced tumor immunoreactivity: increased expression of CD69, CD86 or MHC class II I-ab: or decreased expression of CD206, or the like). Typically, in these methods, the tumor-associated macrophage is disposed in an individual diagnosed with cancer (e.g. a lymphoma or a skin, breast, ovarian, prostate, colorectal or lung cancer): and the individual is undergoing a therapeutic regimen comprising the administration of a chemotherapeutic agent. Optionally, the monoamine oxidase A inhibitor comprises at least one of phenelzine; moclobemide; clorgyline; pirlindole; isocarboxazid; tranylcypromide; iproniazid; caroxazone; befloxatone; brofaromine; cimoxatone; eprobemide; esuprone; metraindol; or toloxatone, for example one of these compounds disposed within a nanoparticle. These methods of the invention can introduce a monoamine oxidase A inhibitor into an environment in which tumor-associated macrophages are disposed in combination with a variety of different chemotherapeutic agents such as antibodies. Optionally for example, a method of the invention introduces at least one immune checkpoint inhibitor chemotherapeutic agent selected to affect CTLA-4 or a PD-1/PD-L1 blockade. In certain embodiments, the checkpoint inhibitor comprises a CTLA-4 blocking antibody, an anti-PD-1 blocking antibody and/or an anti-PD-L1 blocking antibody. In other embodiments, the chemotherapeutic agent comprises carboplatin, cisplatin, paclitaxel, doxorubicin, docetaxel, cyclophosphamide, etoposide, fluorouracil, gemcitabine, methotrexate, erlotinib, imatinib mesylate, irinotecan, sorafenib, sunitinib, topotecan, vincristine, vinblastine, or the like.

Yet another embodiment of the invention is a method of treating a cancer (e.g. a lymphoma or a skin, breast, ovarian, prostate, colorectal or lung cancer) in an individual comprising administering to the individual a monoamine oxidase A inhibitor: wherein amounts of the monoamine oxidase A inhibitor administered to the individual are selected to be sufficient to modulate the phenotype of tumor-associated macrophages in the individual (e.g. wherein modulation of the phenotype comprises decreased levels of intracellular reactive oxygen species: enhanced tumor immunoreactivity: increased expression of CD69, CD86 or MHC class II I-ab: or decreased expression of CD206). Optionally, the monoamine oxidase A inhibitor comprises at least one of phenelzine: moclobemide: clorgyline: pirlindole: isocarboxazid: tranylcypromide: iproniazid: caroxazone: befloxatone: brofaromine: cimoxatone: eprobemide: esuprone: metraindol: or toloxatone, for example one of these compounds disposed within a nanoparticle. In certain embodiments, the individual is undergoing a therapeutic regimen comprising the administration of at least one chemotherapeutic agent. Some embodiments of the invention include methods of administering monoamine oxidase A inhibitor to the individual in combination with a chemotherapeutic agent. Optionally for example, a method of the invention includes administering a monoamine oxidase A inhibitor to the individual in combination with at least one immune checkpoint inhibitor chemotherapeutic agent selected to affect CTLA-4 or a PD-1/PD-L1 blockade. In certain embodiments, the checkpoint inhibitor comprises a CTLA-4 blocking antibody, an anti-PD-1 blocking antibody and/or an anti-PD-L1 blocking antibody. In other embodiments of the invention, the chemotherapeutic agent comprises carboplatin, cisplatin, paclitaxel, doxorubicin, docetaxel, cyclophosphamide, etoposide, fluorouracil, gemcitabine, methotrexate, erlotinib, imatinib mesylate, irinotecan, sorafenib, sunitinib, topotecan, vincristine, vinblastine, or the like.

In methods of the invention, the monoamine oxidase inhibitor is administered in a therapeutically effective amount/dose (e.g. an amount sufficient to modulate the phenotype of tumor-associated macrophages in a patient), which may vary depending upon a variety of factors including the specific monoamine oxidase inhibitor; the age, body weight, general health, sex, and diet of the patient: the mode and time of administration: the rate of excretion: the drug combination: the severity of the particular disorder or condition: and the subject undergoing therapy. The pharmacology of monoamine oxidase inhibitors is well known in the art and, using this information in combination with the disclosure presented herein (e.g. the disclosure below and FIG. Z), doses of such inhibitors can be tailored to the individual subject (e.g. in order to modulate the phenotype of tumor-associated macrophages), as is understood and determinable by one skilled in the relevant arts (see, e.g., Monoamine Oxidase Inhibitors: Clinical Pharmacology, Benefits, and Potential Health Risks (Pharmacology—Research, Safety Testing and Regulation) UK ed. Edition by Sushil K. Sharma (Editor): Berkowet al., eds.: Yamada et al., Clinical Pharmacology of MAO Inhibitors: Safety and Future, NeuroToxicology Volume 25, Issues 1-2, January 2004 Pages 215-221: McDaniel et al., Clinical pharmacology of monoamine oxidase inhibitors: Clin Neuropharmacol. 1986:9(3):207-34. doi: 10.1097/00002826-198606000-00001 Hermann et al., Current place of monoamine oxidase inhibitors in the treatment of depression: CNS Drugs. 2013 Oct.:27(10):789-9: as well as The Merck Manual, 16^{th} edition, Merck and Co., Rahway, N.J., 1992: Goodman et al., eds., Goodman and Gilman's The Pharmacological Basis of Therapeutics, 10^{th} edition, Pergamon Press, Inc., Elmsford, N.Y., (2001): Avery's Drug Treatment: Principles and Practice of Clinical Pharmacology and Therapeutics, 3rd edition, ADIS Press, LTD., Williams and Wilkins, Baltimore, Md. (1987), Ebadi, Pharmacology, Little, Brown and Co., Boston, (1985): Osolci al., eds., Remington's Pharmaceutical Sciences, 18^{th} edition, Mack Publishing Co., Easton, Pa. (1990): Katzung, Basic and Clinical Pharmacology, Appleton and Lange, Norwalk, Conn. (1992)). The total dose required for each treatment can be administered by multiple doses or in a single dose over the course of a day, or a week or a month, if desired.

Further aspects and embodiments of the invention are discussed in the following sections.

### Results

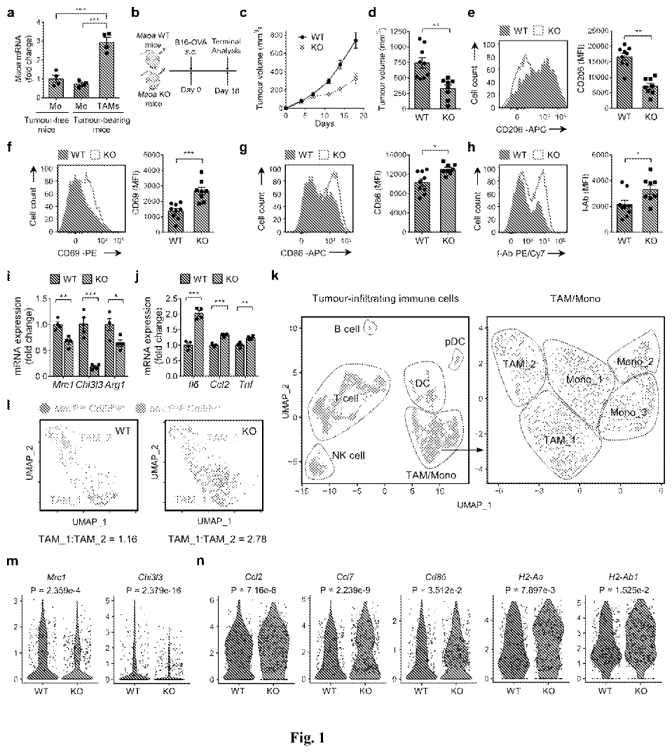

MAO-A-deficient mice show reduced tumour growth associated with altered TAM polarization
In a search for new molecules regulating TAM reprogramming, we inoculated C57BL/6J mice with syngeneic B16-OVA melanoma tumours, isolated TAMs, and assessed TAM gene expression profiles. Monocytes isolated from tumour-free and tumour-bearing mice were included as controls. In addition to changes in classical genes involved in regulating macrophage immune responses, we observed the induction of a Maoa gene in TAMs (FIG. 1a), suggesting that MAO-A may be involved in modulating TAM activities.

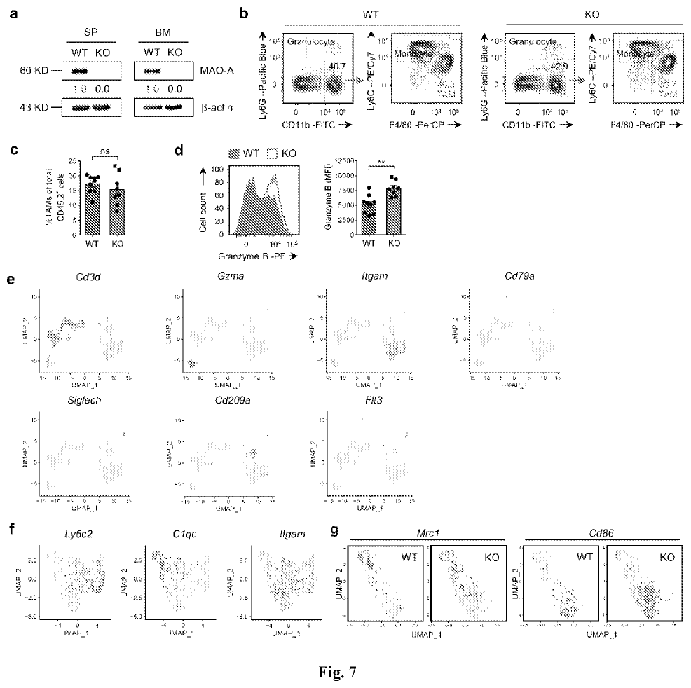

To study the role of MAO-A in antitumour immunity in vivo, we used MAO-A-deficient mice that carry a hypomorphic MAO-A mutant⁵⁶. Although a degree of Maoa expression leakage in the brain had been previously reported in these mice⁵⁶, analysis of their immune system showed a nearly complete ablation of MAO-A expression in major lymphoid organs including spleen and bone marrow (BM) (FIG. 7a). Since we focused on immune cells in this study, we denote these mice as Maoa knockout (KO) mice. When challenged with B16-OVA melanoma cells (FIG. 1b), tumour growth in Maoa KO mice was significantly suppressed compared to that in Maoa wildtype (WT) mice (FIG. 1c,d). Although similar levels of TAMs (gated as CD45.2⁺CD11b⁺Ly6G⁻Ly6C^{-/low}F4/80⁺ cells) were detected in Maoa WT and Maoa KO mice (FIG. 7b,c), compared to their WT counterparts, TAMs isolated from Maoa KO mice exhibited a less immunosuppressive phenotype, indicated by their decreased expression of immunosuppressive markers (i.e., CD206: FIG. 1e), and their increased expression of immunostimulatory molecules (i.e., CD69, CD86, and MHC class II I-Ab: FIG. 1f-h). Further analysis showed that TAMs from Maoa KO mice expressed reduced levels of immunosuppression-associated genes (i.e., Mrc1, Chi313, and Arg1: FIG. 1i) and increased levels of pro-inflammatory cytokine genes (i.e., I16, Tnfa, and Cc12: FIG. 1j). Corresponding to the altered TAM polarization in Maoa KO mice, tumour-infiltrating CD8⁺ T cells in these mice showed enhanced activation (i.e., increased production of Granzyme B: FIG. 7d). Single-cell RNA sequencing (scRNAseq) analysis was performed on tumour infiltrating immune cells isolated from Maoa WT and Maoa KO mice (FIG. 1k and FIG. 7e,). UMAP analysis of extracted TAMs showed a reduced immunosuppressive phenotype in Maoa KO mice, with an increased ratio of Mrc1^{low}Cd86^{high} cells to Mrc1^{high}Cd86^{low} cells (FIG. 1l and FIG. 7g). Gene expression profile analysis confirmed a reduction of TAMs expressing immunosuppressive genes (i.e., Mrc1 and Chi313: FIG. 1m) and an enrichment of TAMs expressing immunostimulatory genes (i.e., Cc12, Cc17, Cd86, H2-Aa, and H2-Ab1: FIG. 1n) in Maoa KO mice. These data strongly indicate that MAO-A is involved in regulating TAM polarization thereby modulating antitumour immunity.

MAO-A Directly Regulates TAM Polarization and Influences TAM-Associated T Cell Antitumour Reactivity

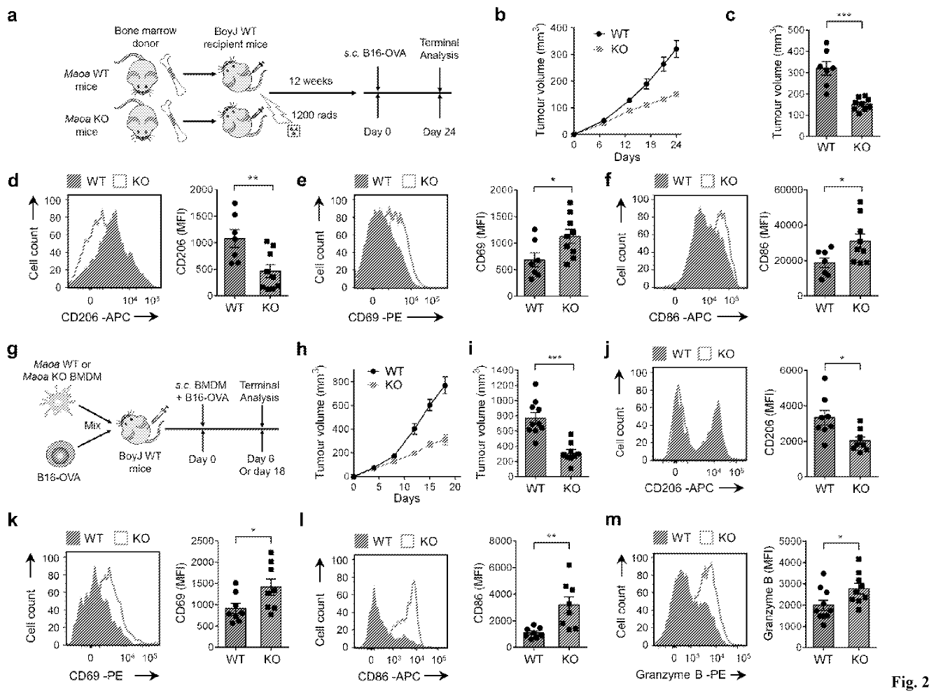

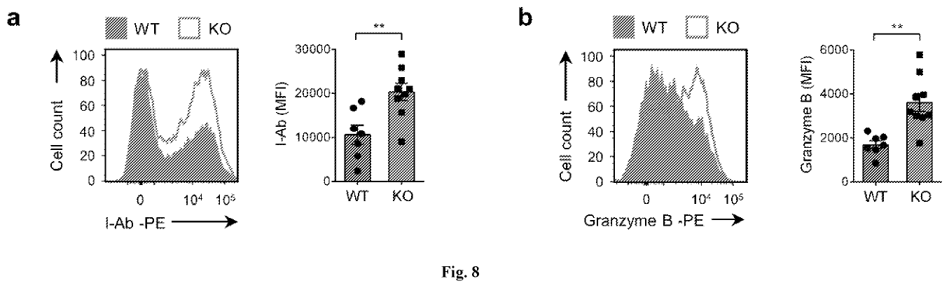

In our Maoa KO mice tumour challenge study, MAO-A deficiency impacted both immune and non-immune cells (FIG. 1b). To determine whether MAO-A directly regulates immune cells, we conducted a BM transfer experiment wherein BM cells harvested from Maoa WT or KO mice were adoptively transferred into BoyJ (CD45.1) WT recipient mice followed by B16-OVA tumour challenge (FIG. 2a). In this experiment, MAO-A deficiency comparison was confined to immune cells. MAO-A deficiency in immune cells resulted in suppressed tumour growth (FIG. 2b,c), altered TAM polarization (i.e., downregulation of immunosuppressive markers such as CD206, FIG. 2d: and upregulation of immunostimulatory markers such as CD69, CD86, and MHC class II I-Ab: FIG. 2e,f and FIG. 8a), and enhanced tumour-infiltrating CD8⁺ T cell activation (i.e., increased production of cytotoxic molecules such as Granzyme B: FIG. 8b), indicating that MAO-A directly regulates immune cell antitumour activity, in particular TAM polarization and T cell antitumour reactivity.

To further study whether MAO-A acts as a macrophage autonomous factor directly regulating TAM polarization and thereby influencing antitumour immunity, we performed a macrophage adoptive transfer tumour experiment. BM cells were harvested from Maoa WT and KO mice then cultured into bone marrow-derived macrophages (BMDMs). These Maoa WT or KO BMDMs were then mixed with B16-OVA melanoma cells and subcutaneously (s.c.) injected into BoyJ WT recipient mice to establish solid tumours (FIG. 2g). In this study, MAO-A deficiency comparison was confined to TAMs. Suppressed tumour growth (FIG. 2h,i), downregulated expression of TAM immunosuppressive markers (i.e., CD206: FIG. 2j), upregulated expression of TAM immunostimulatory markers (i.e., CD69 and CD86: FIG. 2k,l), and enhanced tumour-infiltrating CD8⁺ T cell reactivity (i.e., increased production of Granzyme B: FIG. 2m) were observed in mice receiving Maoa KO BMDMs. Collectively, these in vivo studies demonstrate that MAO-A acts as an autonomous factor directly regulating TAM polarization, and thereby influencing T cell antitumour reactivity and impacting tumour growth.

### MAO-A Promotes Macrophage Immunosuppressive Polarization

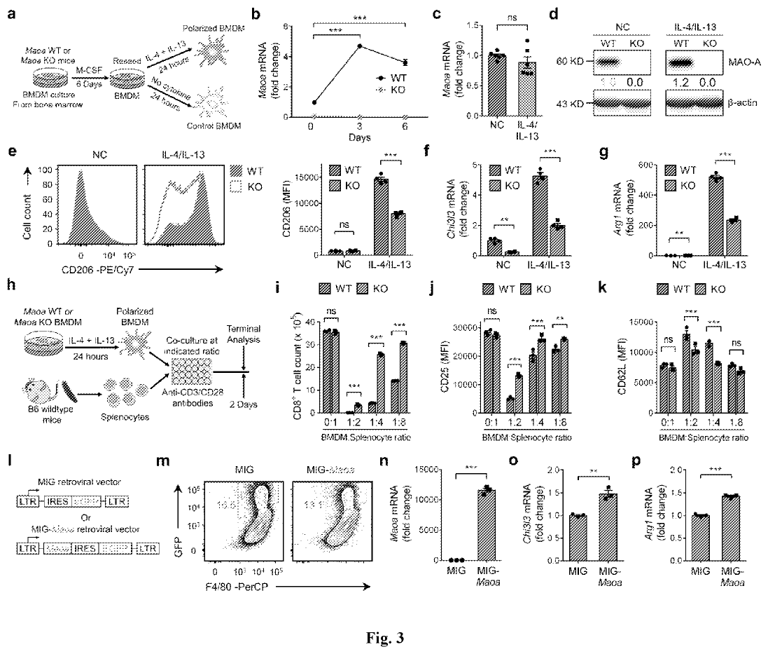

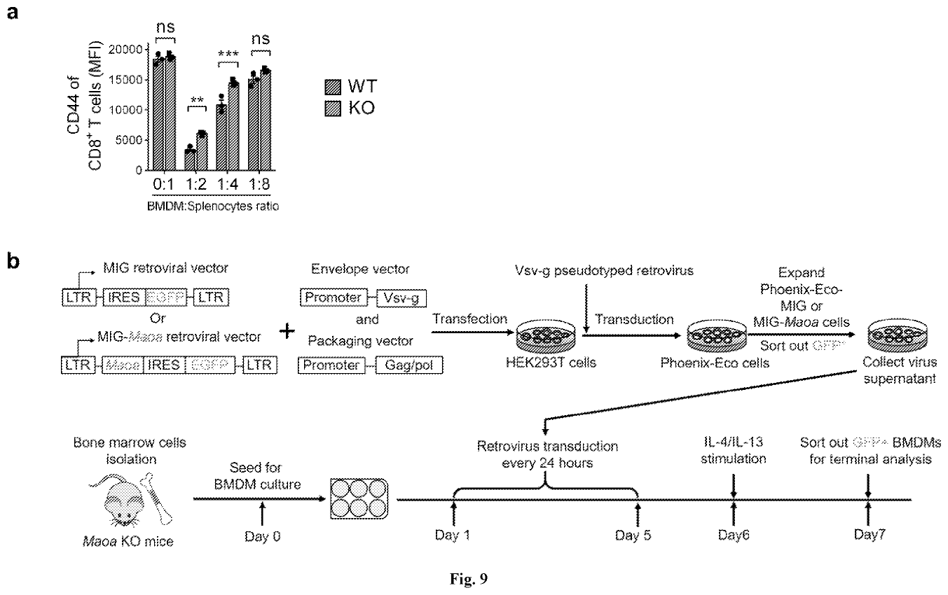

To study MAO-A regulation of macrophage polarization, we cultured Maoa WT and KO BMDMs in vitro and polarized these macrophages toward an immunosuppressive phenotype by adding anti-inflammatory stimuli (i.e., IL-4 and IL-13: FIG. 3a). We observed a sharp induction of Maoa mRNA expression in Maoa WT BMDMs during macrophage development, that remained high post-IL-4/IL-13 stimulation (FIG. 3b,c). MAO-A expression was undetectable in Maoa KO BMDMs, confirming their Maoa-deficiency genotype (FIG. 3b,d). Compared to their wildtype counterpart, Maoa KO macrophages displayed a less immunosuppressive phenotype under IL-4/IL-13 stimulation, evidenced in their reduced expression of immunosuppressive markers (i.e., CD206: FIG. 3e) and signature genes (i.e., Chi313 and Arg1: FIG. 3f,g). When tested in a macrophage/T cell co-culture assay (FIG. 3h), in agreement with their less immunosuppressive phenotype, IL-4/IL-13-polarized Maoa KO macrophages exhibited impaired suppression of wildtype CD8⁺ T cells under anti-CD3/CD28 stimulation, shown as their attenuated inhibition of CD8⁺ T cell proliferation (FIG. 3i) and activation marker expression (i.e., upregulation of CD25 and CD44, and downregulation of CD62L: FIG. 3j,k and FIG. 9a).

To verify whether MAO-A deficiency directly contributed to the alleviated immunosuppressive polarization of Maoa KO macrophages, we performed a rescue experiment. We constructed a MIG-Maoa retroviral vector, used this vector to transduce Maoa KO BMDMs, and achieved overexpression of MAO-A in these macrophages (FIG. 3l-n, and FIG. 9b). MAO-A overexpression significantly exacerbated the immunosuppressive phenotype of IL-4/IL-13-stimulated Maoa KO BMDMs (i.e., upregulation of immunosuppressive signature genes such as Chi313 and Arg1: FIG. 3o,p). Taken together, these results indicate that MAO-A acts as an autonomous factor promoting macrophage immunosuppressive polarization under anti-inflammatory stimuli.

### MAO-A Promotes Macrophage Immunosuppressive Polarization Via ROS Upregulation

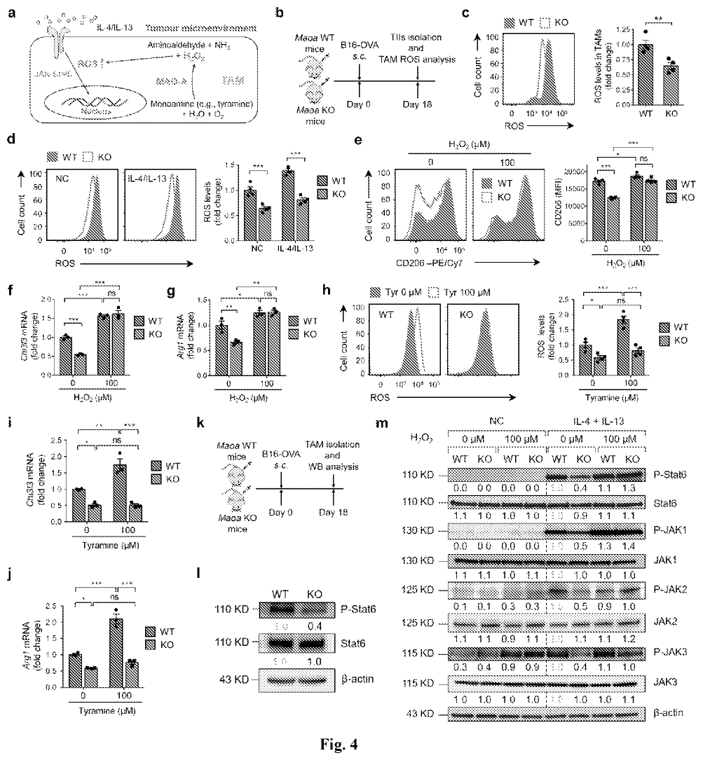

Next, we sought to investigate the molecular mechanisms regulating MAO-A promotion of macrophage immunosuppressive polarization. It has been reported that intracellular reactive oxygen species (ROS: hence, oxidative stress) elicit macrophage immunosuppressive features^{57, 58, 59, 60, 61}. MAO-A catalyzes the oxidative deamination of monoamines, thereby generating hydrogen peroxide (H₂O₂) as a byproduct that can increase intracellular ROS levels. We therefore speculated that MAO-A might promote TAM immunosuppressive polarization in TME via upregulating ROS levels in TAMs (FIG. 4a).

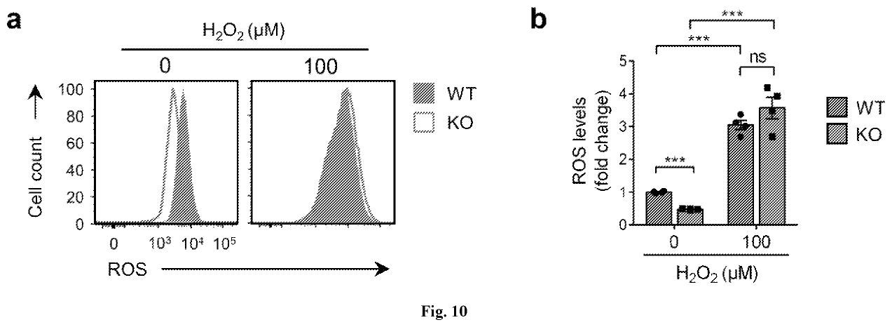

To test this hypothesis, we directly measured ROS levels in TAMs isolated from Maoa WT and KO mice bearing B16-OVA tumours and detected significantly lower levels of ROS in Maoa KO TAMs (FIG. 4b,c). Measurement of ROS levels in in vitro-cultured Maoa WT and KO BMDMs also showed reduced levels of ROS in Maoa KO BMDMs, with or without IL-4/IL-13 stimulation, in agreement with the in vivo TAM results (FIG. 4d). Supplementing H₂O₂ to IL-4/IL-13-stimulated Maoa WT and KO BMDMs elevated their intracellular ROS to similar levels (FIG. 10a,b) and eliminated their differences in expression of immunosuppressive markers (i.e., CD206. FIG. 4e) and signature genes (i.e., Chi313 and Arg1: FIG. 4f,g).

On the other hand, supplementation of tyramine, a substrate of MAO-A, increased ROS levels and upregulated the expression of immunosuppressive genes (i.e., Chi313 and Arg1) in Maoa WT BMDMs but not in Maoa KO BMDMs (FIG. 4h-j). Taken together, these data indicate that MAO-A regulates macrophage immunosuppressive polarization via modulating macrophage intracellular ROS levels.

The JAK-Stat6 signaling pathway plays a key role in mediating IL-4/IL-13-induced immunosuppressive polarization of TAMs in TME^{62, 63}. After IL-4/IL-13 stimulation, JAK is phosphorylated and subsequently phosphorylates Stat6: phosphorylated Stat6 dimerizes and migrates to the nucleus, where it binds to the promoters of IL-4 and IL-13 responsive genes including those involved in macrophage immunosuppressive functions^{64, 65}. ROS has been reported to promote JAK and Stat6 phosphorylation in a variety of cell types^{61, 66, 67, 68, 69, 70, 71}. Since we observed decreased ROS levels in Maoa KO macrophages compared to those in Maoa WT macrophages (FIG. 4b,c), we postulated that MAO-A may impact macrophage polarization through upregulating ROS levels and thereby sensitizing the JAK-Stat6 signaling pathway. Indeed, direct analysis of TAMs isolated from B16-OVA tumour-bearing Maoa WT and Maoa KO mice confirmed that compared to wildtype TAMs, MAO-A-deficient TAMs showed reduced Stat6 activation (i.e., reduced Stat6 phosphorylation: FIG. 4k,l). Further analysis of IL-4/IL-13-induced JAK-Stat6 signaling pathway in Maoa KO BMDMs compared to that in Maoa WT BMDMs showed significantly reduced JAK-Stat6 signaling (i.e., reduced JAK1, JAK2, JAK3, and Stat6 phosphorylation: FIG. 4m). Supplementing H₂O₂ to IL-4/IL-13-stimulated Maoa WT and KO BMDMs increased their JAK-Stat6 signaling to similar levels (i.e., comparable JAK1, JAK2, JAK3, and Stat6 phosphorylation: FIG. 4m), corresponding to their comparable high levels of ROS (FIG. 10a,b). These data indicate that MAO-A promotes macrophage immunosuppressive polarization via ROS-sensitized JAK-Stat6 pathway activation.

Collectively, these in vivo and in vitro data support a working model that MAO-A promotes TAM immunosuppressive polarization in TME, at least partly through upregulating TAM intracellular ROS levels and thereby enhancing the IL-4/IL-13-induced JAK-Stat6 signaling pathway.

### MAO-A Blockade for Cancer Immunotherapy-Syngeneic Mouse Tumour Model Studies

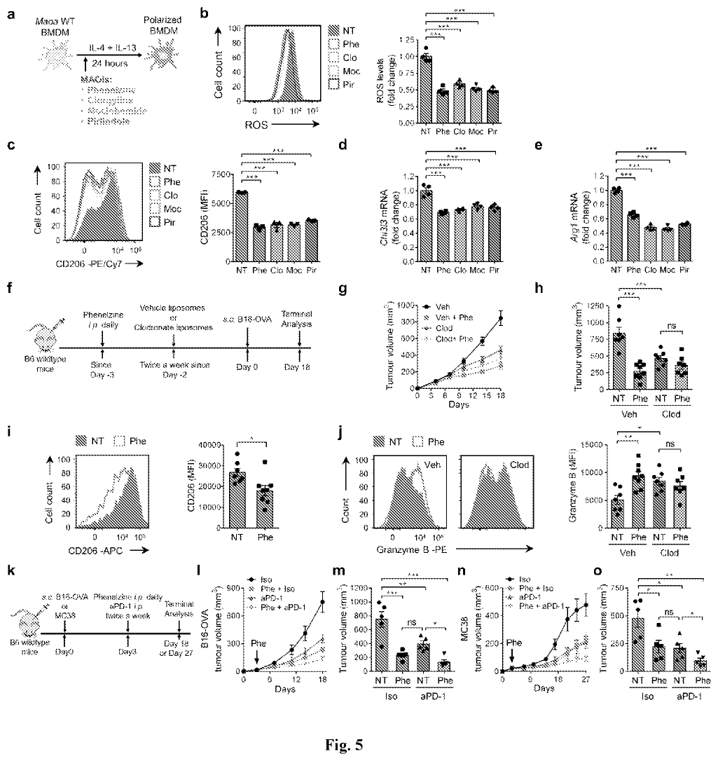

The identification of MAO-A as a key regulator of TAM immunosuppressive polarization makes MAO-A a promising new drug target for cancer immunotherapy. Because of the known functions of MAO-A in the brain, small molecule MAOIs have been developed and clinically utilized for treating various neurological disorders, making it a highly feasible and attractive approach to repurpose these established MAOI drugs for cancer immunotherapy^{51, 72}. In an in vitro WT BMDM IL-4/IL-13-induced polarization culture (FIG. 5a), addition of multiple MAOIs efficiently reduced ROS levels in BMDMs (FIG. 5b) and suppressed their immunosuppressive polarization, evidenced by their decreased expression of immunosuppressive markers (i.e., CD206: FIG. 5c) and immunosuppressive genes (i.e., Chi313 and Arg1: FIG. 5d,e). Notably, the MAOIs that we tested include phenelzine, clorgyline, mocolobemide, and pirlindole, covering the major categories of established MAOIs classified on the basis of whether they are nonselective or selective for MAO-A, and whether their effect is reversible (FIG. 5a)^{51, 54, 73}. Among these MAOIs, phenelzine (trade name: Nardil) is clinically available in the United States⁷². In the following studies, we chose phenelzine as a representative to study the possibility of repurposing MAOIs for cancer immunotherapy, using two syngeneic mouse tumour models: a B16-OVA melanoma model and a MC38 colon cancer model⁷⁴.

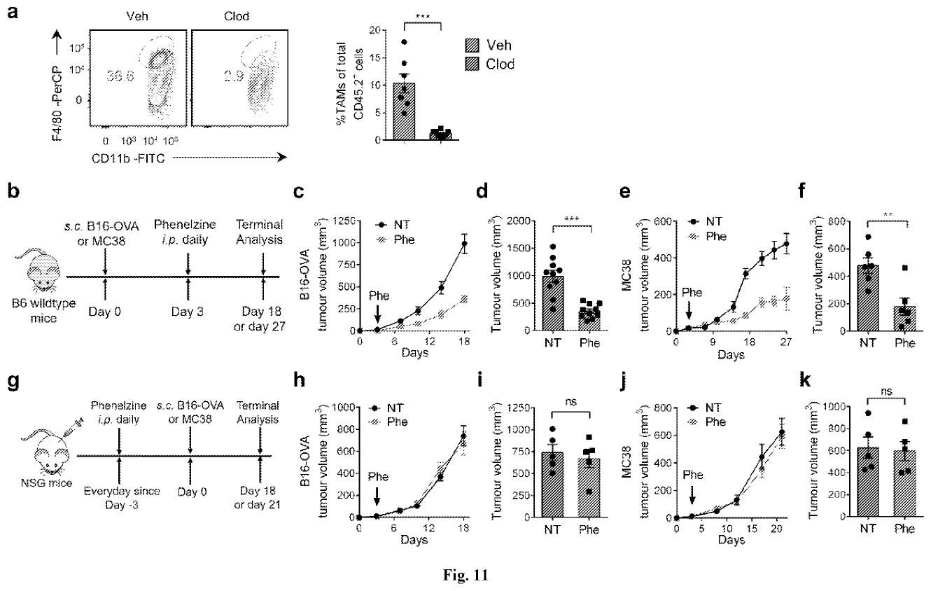

First, we studied the therapeutic potential of phenelzine in a B16-OVA tumour prevention model (FIG. 5f). Phenelzine treatment effectively suppressed B16-OVA tumour growth in B6 wildtype mice (FIG. 5g,h). No tumour growth difference was observed when we depleted TAMs in experimental mice via a clodronate liposome treatment, indicating that phenelzine suppressed tumour growth via modulating TAMs (FIG. 5g,h and FIG. 11a). Correspondingly, TAMs isolated from phenelzine-treated mice displayed a less immunosuppressive phenotype (i.e., decreased expression of CD206: FIG. 5i) that was correlated with an enhanced antitumour reactivity of tumour-infiltrating CD8⁺ T cells (i.e., increased production of Granzyme B: FIG. 5j) in these mice. Further studies showed that phenelzine treatment also effectively suppressed the progression of pre-established solid tumours in both B16-OVA and MC38 models (FIG. 11b-f).

Next, we evaluated the potential of phenelzine for combination therapy, in particular combining with other ICB therapies such as PD-1/PD-L1 blockade therapy (FIG. 5k). Although most ICB therapies target CD8⁺ T cells, these cells are in fact closely regulated by TAMs in the TME, making targeting TAMs another potential avenue for immunotherapy^{14, 39}. In both B16-OVA and MC38 tumour models, phenelzine treatment significantly suppressed the progression of pre-established solid tumours at a level comparable to the anti-PD-1 treatment: importantly, the combination of phenelzine and anti-PD-1 treatments yielded synergistic tumour suppression efficacy (FIG. 5l-o). These tumour suppression effects of phenelzine were due to immunomodulation but not direct tumour inhibition, because phenelzine treatment did not suppress the growth of B16-OVA and MC38 tumours in immunodeficient NSG mice (FIG. 11g-k).

Collectively, these syngeneic mouse tumour model studies provided proof-of-principle evidence for the cancer immunotherapy potential of MAOIs via targeting TAM reprogramming and thereby enhancing antitumour T cell responses.

### MAO-A Blockade for Cancer Immunotherapy-Human TAM and Clinical Data Correlation Studies

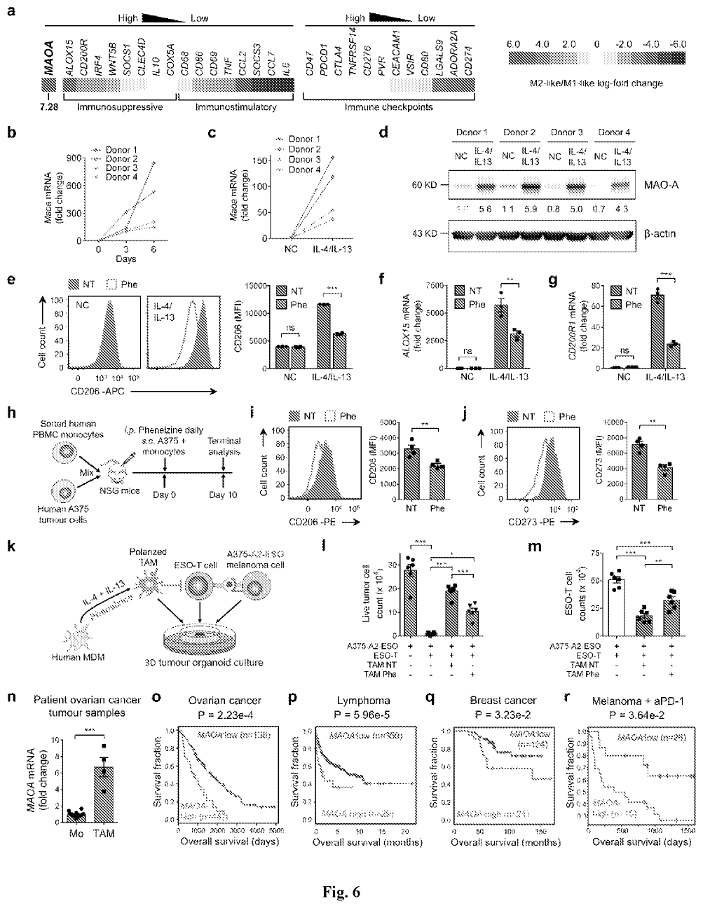

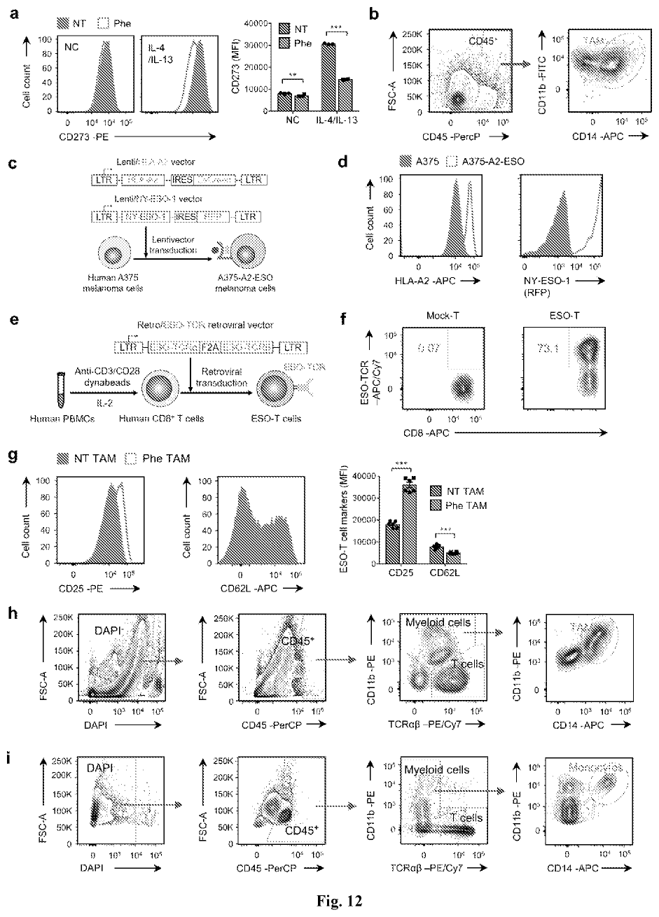

To explore the translational potential of MAO-A blockade therapy, we first studied MAO-A regulation of human macrophage polarization. Using a Tumour Immune Dysfunction and Exclusion (TIDE) computational method⁷⁵, we analyzed the gene expression signatures of in vitro cultured immunostimulatory MI-like and immunosuppressive M2-like human monocyte-derived macrophages (MDMs) (GSE35449)⁷⁶. Interestingly, among all immune checkpoint and immune suppressive genes examined, MAOA ranked as the top gene with the most dramatically elevated expression in M2-like MDMs (i.e., 7.28 M2/M1 log-fold change: FIG. 6a), suggesting a possible role of MAO-A in promoting human macrophage immunosuppressive polarization. Time-course analysis of MDM culture confirmed an upregulation of MAO-A gene and protein expression during macrophage differentiation that was further upregulated post IL-4/IL-13-induced immunosuppressive polarization (FIG. 6b-d). Blockade of MAO-A using phenelzine significantly inhibited IL-4/IL-13-induced immunosuppressive polarization of MDMs, evidenced by their decreased expression of immunosuppressive markers (i.e., CD206 and CD273: FIG. 6e and FIG. 12a) and signature genes (i.e., ALOX15 and CD200R1: FIG. 6f,g). Collectively, these in vitro data suggest that MAO-A is highly expressed in human macrophages especially during their immunosuppressive polarization, and that MAO-A blockade has the potential to reprogram human macrophage polarization.

To directly evaluate whether MAO-A blockade could reprogram human TAM polarization in vivo, we established a human tumour/TAM xenograft NSG mouse model. A375 human melanoma cells were mixed with monocytes sorted from healthy donor peripheral blood mononuclear cells (PBMCs), and s.c. injected into NSG mice to form solid tumours, with or without phenelzine treatment after inoculation (FIG. 6h). Phenelzine treatment effectively suppressed immunosuppressive polarization of human TAMs (gated as hCD45⁺hCD11b⁺hCD14⁺: FIG. 12b), supported by their decreased expression of immunosuppressive markers (i.e., CD206 and CD273: FIG. 6i,j).

Next, we studied whether MAO-A blockade-induced human TAM reprogramming could impact human T cell antitumour reactivity, using a 3D human tumour/TAM/T cell organoid culture (FIG. 6k). NY-ESO-1, a well-recognized tumour antigen commonly expressed in a large variety of human cancers⁷⁷, was chosen as the model tumour antigen. An A375 human melanoma cell line was engineered to co-express NY-ESO-1 as well as its matching MHC molecule, HLA-A2, to serve as the human tumour target (denoted as A375-A2-ESO: FIG. 12c,d). NY-ESO-1-specific human CD8⁺ T cells were generated by transducing healthy donor peripheral blood CD8⁺ T cells with a Retro/ESO-TCR retroviral vector encoding an NY-ESO-1 specific TCR (clone 3A1: denoted as ESO-TCR): the resulting T cells, denoted as ESO-T cells, expressed ESO-TCRs and specifically targeted A375-A2-ESO tumour cells, thereby modeling the tumour-specific human CD8⁺ T cells (FIG. 12e,f). Human MDMs were cultured from healthy donor PBMCs, followed by IL-4/IL-13 stimulation to induce immunosuppressive polarization in the presence or absence of phenelzine treatment (FIG. 6k). The A375-A2-ESO human melanoma cells, ESO-T cells, and IL-4/IL-13-polarized MDMs were mixed at a 2:2:1 ratio and placed in a 3D tumour organoid culture mimicking TME (FIG. 6k). IL-4/IL-13-polarized MDMs effectively suppressed ESO-T cell-mediated killing of A375-A2-ESO tumour cells: this immunosuppressive effect was largely alleviated by phenelzine treatment during MDM polarization (FIG. 6l). Accordingly, ESO-T cells co-cultured with phenelzine-treated MDMs, compared to those co-cultured with non-phenelzine-treated MDMs, showed an enhancement in T cell activation (i.e., increased cell number, increased CD25 expression, and decreased CD62L expression: FIG. 6m and FIG. 12g). Collectively, these data suggest that MAO-A blockade-induced human TAM reprogramming has the potential to improve antitumour T cell responses.

To study MAOA gene expression in primary human TAMs, we collected fresh ovarian cancer tumour samples from patients, isolated TAMs (sorted as DAPI-hCD45⁺hCD11b⁺hTCRαβ⁻hCD14⁺ cells: FIG. 12h), and assessed their MAOA gene expression. Primary human monocytes isolated from health donor PBMCs (sorted as DAPI⁻hCD45⁺hCD11b⁺hTCRαβ⁻hCD14+ cells: FIG. 12i) were included as controls. Like mouse TAMs, human TAMs expressed high levels of MAOA gene, confirming MAO-A as a valid drug target in human TAMs (FIG. 1a and FIG. 6n).

Lastly, we conducted clinical data correlation studies to investigate whether intratumoural MAOA gene expression is correlated with clinical outcomes in cancer patients, using the TIDE computational method⁷⁵. Intratumoural MAOA expression level was negatively correlated with patient survival in multiple cancer patient cohorts spanning ovarian cancer (FIG. 6o)⁷⁸, lymphoma (FIG. 6p)⁷⁹, and breast cancer (FIG. 6q)⁸⁰. Moreover, analysis of a melanoma patient cohort receiving anti-PD-1 treatment showed that high levels of intratumoural MAOA expression largely abrogated the survival benefit offered by the PD-1 treatment, suggesting that combining MAO-A blockade therapy with PD-1/PD-L1 blockade therapy may provide synergistic therapeutic benefits through modulating TAM polarization and thereby changing the immunosuppressive TME and improving antitumour immunity (FIG. 6r)⁸¹. Of note, these whole tumour lysate transcriptome data analyses could not localize the MAOA expression to a specific cell type (e.g., TAMs): future studies of quality transcriptome data generated from single cells or sorted TAMs are needed to obtain such information. Nonetheless, the present clinical data correlation studies identified MAO-A as a possible negative regulator of survival in a broad range of cancer patients, including those receiving existing ICB therapies, suggesting MAO-A blockade as a promising avenue for developing new forms cancer therapy and combination therapy.

Taken together, these human TAM and clinical correlation studies confirmed MAO-A as a promising drug target in human TAMs and support the translational potential of MAO-A blockade for cancer immunotherapy through targeting TAM reprogramming.

### DISCUSSION

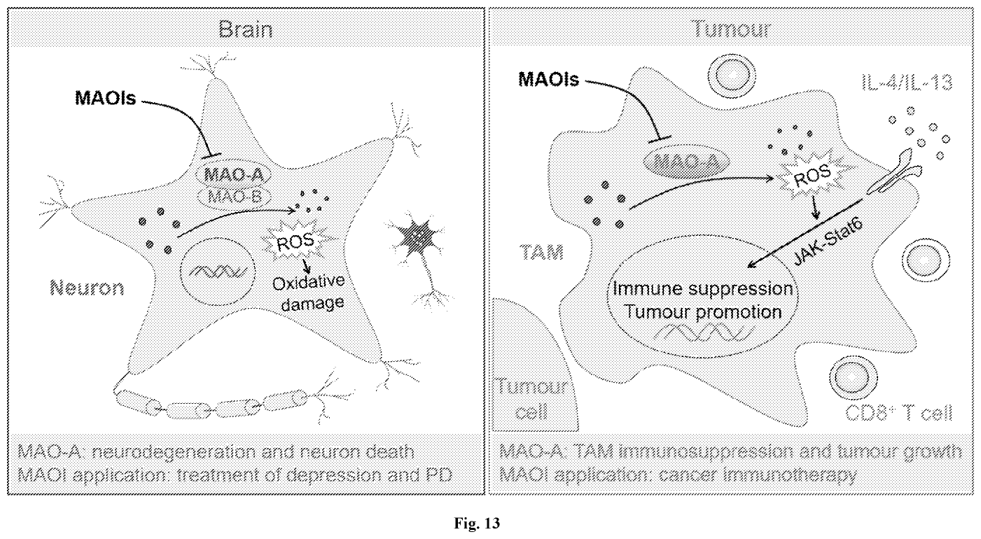

Based on our findings, we propose an “intratumoural MAO-A-ROS axis” model to elucidate the role of MAO-A in regulating TAM immunosuppressive polarization (FIG. 13). Analogous to the well-characterized MAO-A-ROS axis in the brain, where MAO-A controls ROS levels in neurons and thereby modulates neuron degeneration via regulating neuron oxidative stress, the MAO-A-ROS axis in a solid tumour controls ROS levels in TAMs and thereby modulates TAM immunosuppressive polarization via sensitizing the IL-4/IL-13-induced JAK-Stat6 signaling pathway (FIG. 13). The resemblance between these mechanisms is intriguing: from an evolutionary point of view, it makes sense that some critical molecular regulatory pathways are preserved between the nervous and immune systems, considering that both systems are evolved to defend a living organism by sensing and reacting to environmental danger and stress. Indeed, neurons and immune cells share a broad collection of surface receptors, secretory molecules, and signal transducers⁸². In particular, many neurotransmitters/neuropeptides and their synthesis/degradation machineries traditionally considered specific for neurons are expressed in immune cells, although their functions in the immune system are to a large extent still unknown^{83, 84}. Studying these molecules and their regulatory mechanisms may provide new perspectives in tumour immunology and identifying new drug targets for cancer immunotherapies, as exemplified by our current finding of this “MAO-A-ROS axis” regulation of TAM polarization in the TME.

Considering the importance of TAMs in regulating antitumour immunity, there has been considerable efforts in developing cancer therapeutic strategies targeting TAMs. These strategies can be roughly divided into two categories: 1) those which deplete TAMs, and 2) those which alter TAM immunosuppressive activities³⁹. The first category includes strategies targeting TAM recruitment and survival, such as blocking the CCL2-CCR2 axis thereby preventing monocyte mobilization from the bone marrow and recruitment into inflammatory sites, or blocking the CSF1-CSF1R axis thereby inducing apoptosis of TAMs, or blocking the CXCL12-CXCR4 and angiopoietin 2 (ANG2)-TIE2 axes thereby depleting TIE2V macrophages that are critical for tumour angiogenesis^{19, 39, 85}. However, an intrinsic downside of depleting TAMs is the loss of their innate immunostimulatory role as the primary phagocytes and professional antigen-presenting cells (APCs) in solid tumours. Reprogramming or repolarizing immunosuppressive TAMs towards an immunostimulatory phenotype therefore can be an attractive direction: this second category of TAM-repolarizing strategies includes those reprogramming TAMs via CD40 agonists, HDAC inhibitors, PI3Kγ inhibitors, and creatine^{39, 40, 86, 87, 88}. Many of these TAM reprogramming strategies are currently under active clinical evaluation³⁹. Notably, CD40 agonists work through activating CD40L-downstream NF-kB pathway^{87, 89}: HDAC inhibitors work through altering histone modifications^{86, 90, 91}: PI3Kγ inhibitors work through stimulating NF-κB activation while inhibiting C/EBPβ activation^{88, 92, 93}: and creatine uptake works through regulating cytokine responses⁴⁰. Our discovery of MAO-A as a critical regulator of TAM polarization through modulating oxidative stress provides a new drug target and a new mechanism of action (MOA) for expanding TAM-repolarizing strategies.

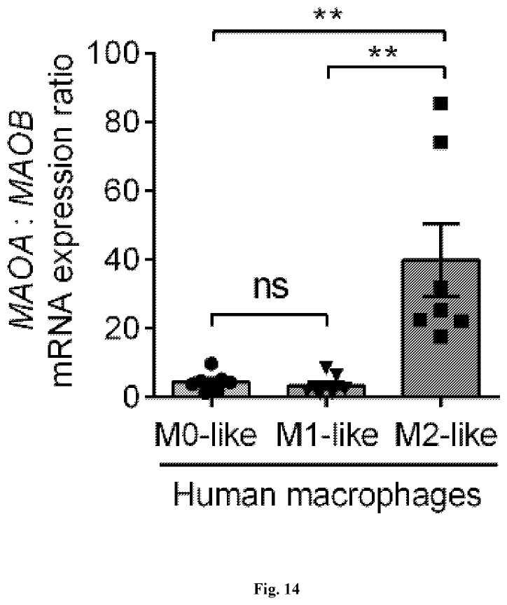

Compared to many new therapeutic candidates, MAO-A is unique in that it is already an established drug target due to its known functions in the brain⁷². In fact, small molecule MAOIs have been developed to block MAO-A enzymatic activity in the brain and are clinically used for treating various neurological disorders⁷². Notably, some MAOIs cross-inhibit the MAO-A isoenzyme MAO-B, that co-expresses with MAO-A in the brain (FIG. 13)⁵¹. However, in human macrophages, especially in M2-like immunosuppressive macrophages, MAO-A is the dominant form (i.e., the expression of MAOA was about 40-fold higher than that of MAOB in M2-like human macrophages: FIG. 14)⁷⁶. In our studies, we tested multiple clinically approved MAOIs (phenelzine, clorgyline, moclobemide, and pirlindole) and demonstrated their efficacy in regulating macrophage ROS levels and immunosuppressive polarization, pointing to the possibility of repurposing these drugs for cancer immunotherapy (FIGS. 5 and 6). Developing new cancer drugs is extremely costly and time-consuming: drug repurposing offers an economic and speedy pathway to novel cancer therapies because approved drugs have known safety profiles and modes of actions and thus can enter the clinic quickly⁹⁴.

MAOIs had been used extensively over two decades after their introduction in the 1950s, but since then their use has declined because of reported side effects and the introduction of other classes of antidepressant drugs⁷². However, these MAOIs side effects were vastly overstated and should be revisited⁷². For instance, a claimed major side effect of MAOIs is the risk of triggering tyramine-induced hypertensive crisis when patients eat tyramine-rich foods such as aged cheese (hence, “cheese effects”): this concern led to cumbersome food restrictions that are now considered largely unnecessary⁷². A transdermal delivery system (Emsam) has also been developed to deliver MAOIs that can largely avoid potential food restrictions⁹⁵. Therefore, interest in MAOIs as a major class of antidepressants is reviving, and repurposing MAOIs for cancer immunotherapy can be an attractive new application of these potent drugs⁷². Moreover, many cancer patients suffer from depression and anxiety: these overwhelming emotional changes can negatively interfere with the quality of life and cancer treatment efficacy of cancer patients⁹⁶. Repurposing MAOIs for cancer immunotherapy thus may provide cancer patients with anti-depression and antitumour dual benefits, making this therapeutic strategy particularly attractive.

Because preclinical evidence largely supports combinatorial approaches being necessary to achieve significant antitumour efficacy, most TAM-targeting strategies currently under clinical evaluation are tested in combination with standard chemotherapy or radiation therapy, or in combination with T cell-directed ICB therapies such as PD-1 or/and PD-L1 blockade therapy³⁹. In our study, we found that MAOI treatment synergized with anti-PD-1 treatment in suppressing syngeneic mouse tumour growth (FIG. 5k-o), and that intratumoural MAOA gene expression levels dictated poor patient survival in melanoma patients receiving anti-PD-1 therapy (FIG. 6r). These data highlight the promise of MAOI treatment as a valuable component for combination cancer therapies.

In summary, here we identified MAO-A as a critical molecule regulating TAM immunosuppressive polarization and thereby modulating antitumour immunity, and demonstrated the potential of repurposing established MAOI antidepressants for cancer immunotherapy. Future clinical studies are encouraged to investigate the clinical correlations between MAOI treatment and clinical outcomes in cancer patients and to explore the possibility of repurposing MAOIs for combination cancer therapies. Meanwhile, the immune regulatory function of MAO-A certainly goes beyond regulating TAM polarization. In Maoa KO mice, we have observed the changes of antitumour responses of multiple immune cells in various syngeneic mouse tumour models. It is also likely that MAO-A regulates immune reactions to other diseases such as infection diseases and autoimmune diseases. Studying the roles of MAO-A in regulating various immune cells under different health and disease conditions will be interesting topics for future research.

### Methods

### Mice

C57BL/6J (B6), B6.SJL-Ptprc^{a}Pepc^{b}/BoyJ (CD45.1, BoyJ), 129S-Maoa^{tw1Slab}/J (Maoa KO)⁵⁶, and NOD.Cg-Prkdc^{scid}I12rg^{tm1Wj1}/SzJ (NSG) mice were purchased from the Jackson Laboratory (Bar Harbor). Maoa KO mice were backcrossed with C57BL/6J mice for more than 9 generations at the University of California, Los Angeles (UCLA). Eight- to twelve-week-old female mice were used for all experiments unless otherwise indicated. Due to ethical reasons, we ended experiments before tumour volume surpassed 1000 mm³. All mice experiments were repeated at least three times unless specifically mentioned. Replicates of each individual experiment was stated in its figure legends. All animals were maintained at the UCLA animal facilities and all animal experiments were approved by the Institutional Animal Care and Use Committee of UCLA.

### Human Tumour Samples

All human samples were obtained following institutional guidelines under protocols approved by the institutional review boards (IRBs) at the UCLA Medical Center. Primary human ovarian cancer tumour samples were obtained from the operating room at the UCLA Medical Center from consenting patients using IRB-approved protocols (IRB #10-000727). Tumour specimens were brought back to the laboratory for further analyses. Detailed patients' information is provided in Table 1, including collection date, age, diagnosis and staging.

### Cell Lines and Viral Vectors

The B16-OVA mouse melanoma cell line and the PG13 retroviral packaging cell line were provided by Dr. Pin Wang (University of South California, CA)⁹⁷. The MC-38 mouse colon adenocarcinoma cell line was provided by Dr. Antoni Ribas (UCLA)⁷⁴. The HEK 293T and Phoenix-ECO retroviral packaging cell lines, the A375 human melanoma cell line, and the L-929 mouse connective tissue cell line were purchased from the American Type Culture Collection (ATCC). The A375-A2-ESO cell line was previously reported⁹⁸. The Phoenix-ECO-MIG, Phoenix-ECO-MIG-Maoa, and PG13-ESO-TCR stable virus producing cell lines were generated in this study. The MIG (MSCV-IRES-GFP) retroviral vector was reported previously^{99, 100, 101}. MIG-Maoa and Retro/ESO-TCR retroviral vectors were generated in this study.

### Syngeneic Mouse Tumour Models

B16-OVA melanoma cells (1×10⁶ per animal) or MC38 colon cancer cells (5×10⁵ per animal) were subcutaneously (s.c.) injected into experimental mice to form solid tumours. In some experiments, mice received intraperitoneal (i.p.) injection of phenelzine (30 mg/kg/day) to block MAO-A activity. In some experiments, mice received i.p. injection of clodronate liposomes (200 μl/animal, twice per week) to deplete TAMs: mice received i.p. injection of vehicle liposomes (200 ρW/animal, twice per week) were included as controls. In some experiments, mice received i.p. injection of anti-mouse PD-1 antibodies (300 μg/animal, twice per week) to block PD-1: mice received i.p. injection of isotype antibodies were included as controls. During an experiment, tumour growth was monitored twice per week by measuring tumour size using a Fisherbrand™ Traceable™ digital caliper (Thermo Fisher Scientific): tumour volumes were calculated by formula 1/2×L×W². At the end of an experiment, solid tumours were collected, and tumour-infiltrating immune cells were isolated for analysis using QPCR, flow cytometry, and/or scRNASeq.

### Bone Marrow (BM) Transfer Mouse Tumour Model

BM cells were collected from femurs and tibias of Maoa WT and Maoa KO donor mice, and were separately transferred into BoyJ (CD45.1) wildtype recipient mice that were preconditioned with whole body irradiation (1,200 rads). Recipient mice were maintained on antibiotic water (Amoxil, 0.25 mg/ml) for 4 weeks after BM transplantation. Periodical bleedings were performed to monitor immune cell reconstitution using flow cytometry. Tumour inoculation started at 12 weeks post BM transfer when recipient mice were fully immune reconstituted. B16-OVA mouse melanoma cells were s.c. injected into recipient mice to form solid tumours (1×10⁶ cells per animal). Tumour growth was monitored twice per week by measuring tumour size using a Fisherbrand™ Traceable™ digital caliper: tumour volumes were calculated by formula 1/2×L×W2. At the end of an experiment, tumour-infiltrating immune cells were isolated for analysis using flow cytometry.

### Syngeneic Mouse Tumour-TAM Co-Inoculation Model

Bone marrow cells were collected from Maoa WT and Maoa KO mice and were cultured in vitro to generate bone marrow-derived macrophages (BMDMs). B16-OVA tumour cells (1×10⁶ cells per mouse) and BMDMs (5×10⁶ cells per mouse) were mixed and s.c. injected into BoyJ mice to form solid tumours. Tumour growth was monitored twice per week by measuring tumour size using a Fisherbrand™ Traceable™ digital caliper: tumour volumes were calculated by formula 1/2×L×W². At the end of an experiment, tumours were collected and tumour-infiltrating immune cells were isolated for analysis using flow cytometry.

### Xenograft Human Tumour-TAM Co-Inoculation Model

Human peripheral blood mononuclear cells (PBMCs) of healthy donors were obtained from the CFAR Gene and Cellular Therapy Core Laboratory at UCLA, without identification information under federal and state regulations. Human monocytes were isolated from healthy donor PBMCs via magnetic-activated cell sorting (MACS) using human CD14 microbeads (Miltenyi Biotec, 130-050-201) followed by fluorescence activated cell sorting (FACS: sorted as hCD45⁺hCD11b⁺hCD14⁺ cells) using a FACSAria II flow cytometer (BD Biosciences). Human A375 melanoma cells (10×10⁶ cells per animal) and purified human monocytes (5×10⁶ cells per animal) were mixed and s.c. injected into NSG mice to form solid tumours. Some experimental animals received i.p. injection of MAOI (phenelzine, 30 mg/kg/day) to block MAO-A activity. At the end of an experiment, tumour-associated immune cells were isolated for analysis using flow cytometry.

### Tumour-Infiltrating Immune Cell (TII) Isolation and Analysis

Solid tumours were collected from experimental mice at the termination of a tumour experiment. Tumours were cut into small pieces and smashed against a 70-μm cell strainer (Corning, 07-201-431) to prepare single cells. Immune cells were enriched through gradient centrifugation with 45% Percoll (Sigma-Aldrich, P4937) at 800 g for 30 mins at 25° C. without braking, followed by treatment with Tris-buffered ammonium chloride buffer to lyse red blood cells according to a standard protocol (Cold Spring Harbor Protocols). The resulting TII isolates were then used for further analysis.

In some experiments, TII isolates were sorted via FACS using a FACSAria II flow cytometer (BD Biosciences) to purify TAMs (sorted as DAPI-CD45.2⁺CD11b⁺Ly6G⁻Ly6C^{-/low}F4/80⁺ cells), which were then subjected to QPCR analysis of Maoa mRNA expression in TAMs.

In some experiments, TII isolates were sorted via FACS using a FACSAria II flow cytometer (BD Biosciences) to purify immune cells (sorted as DAPI⁻CD45.2⁺ cells), which were then subjected to scRNASeq analysis of gene expression profiling of TIIs.

In some experiments, TII isolates were directly analyzed using MACSQuant Analyzer 10 Flow Cytometer (Miltenyi Biotec) to study the cell surface marker expression of TAMs (pre-gated as CD45.2⁺CD11b⁺Ly6G⁻Ly6C^{-/low}F4/80⁺ cells) and the intracellular effector molecule production of CD8⁺ T cells (pre-gated as CD45.2⁺TCRβ⁺CD8⁺ cells).

### Mouse Bone Marrow-Derived Macrophages (BMDM) Culture and Polarization

To generate BMDMs, BM cells were collected from femurs and tibias of Maoa WT mice and Maoa KO mice, and were cultured in C10 medium containing with 20% of L929-conditional medium in a 10-cm dish (2×10⁶ cells per ml: 12 ml per dish) for 6 days. At day 6, the resulting BMDMs were collected and reseeded in a 6-well plate (1×10⁶ cells per ml: 2 ml per well) in C10 medium for 24 hours, in the presence or absence of recombinant murine IL-4 (10 ng/ml) (Peprotech, 200-04) and IL-13 (10 ng/ml) (Peprotech, 200-13) to induce BMDM immunosuppressive polarization.

In some experiments, MAOIs were added to the Maoa WT BMDM polarization culture 30 minutes prior to adding recombinant murine IL-4 and IL-13, to block MAO-A activity during BMDM polarization. MAOIs studied were phenelzine (Phe, 20 μM) (Sigma-Aldrich), clorgyline (Clo, 20 μM) (Sigma-Aldrich), moclobemide (Moc, 200 μM) (Sigma-Aldrich), and pirlindole (Pir, 20 μM) (R&D Systems). At 24 hours after IL-4/IL-13 stimulation, BMDMs were collected for analysis.

In some experiments, H₂O₂ (100 μM) were added to the Maoa WT and Maoa KO BMDM polarization culture 30 minutes prior to adding recombinant murine IL-4 and IL-13. At 30 minutes after IL-4/IL-13 stimulation, BMDMs were collected for WB analysis: at 24 hours after IL-4/IL-13 stimulation, BMDMs were collected for flow cytometry and QPCR analysis.

In some experiments, tyramine (100 μM) (Sigma-Aldrich, T90344) were added to the Maoa WT and Maoa KO BMDM polarization culture 30 minutes prior to adding recombinant murine IL-4 and IL-13. At 24 hours after IL-4/IL-13 stimulation, BMDMs were collected for flow cytometry and QPCR analysis.

### Macrophage Suppressive Function Assay

IL-4/IL-13 polarized Maoa WT and Maoa KO BMDMs were mixed with splenocytes harvested from B6 wildtype mice at 0:1, 1:2, 1:4, or 1:8 ratio, then cultured in a 24-well plate in C10 medium (1×10⁶ splenocytes/ml/well), in the presence of plate-bound anti-mouse CD3ε (5 μg/ml) and soluble anti-mouse CD28 (1 μg/ml) for 2 days. At the end of a culture, cells were collected for flow cytometry analysis.

### MIG-Maoa Retroviral Vector Construction, Production, and Macrophage Transduction

MIG retroviral vector was reported previously^{99, 100, 101}. Codon-optimized Maoa cDNA (synthesized by IDT) was inserted into a MIG retroviral vector to generate the MIG-Maoa retroviral vector. Vsv-g-pseudotyped MIG and MIG-Maoa retroviruses were produced using HEK 293T virus packaging cells following a standard calcium precipitation method^{100, 101}, and then were used to transduce Phoenix-ECO cells to generate stable cell lines producing ECO-pseudotyped MIG or MIG-Maoa retroviruses (denoted as Phoenix-ECO-MIG and Phoenix-ECO-MIG-Maoa cell lines, respectively). For virus production, Phoenix-ECO-MIG and Phoenix-ECO-MIG-Maoa cells were seeded at a density of 0.8×10⁶ cells per ml in D10 medium, and cultured in a 15-cm dish (30 ml per dish) for 2 days. Virus supernatants were then collected and used for macrophage transduction.

BM cells harvested from Maoa WT and Maoa KO mice were cultured in a 6-well plate in C10 medium containing 20% L929-conditional medium (4×10⁶ cells/2 ml/well) for 6 days, to differentiate into BMDMs. From day 1 to day 5, cells were spin-infected daily with virus supernatants supplemented with polybrene (10 μg/ml) at 660 g at 30° C. for 90 minutes. At day 6, recombinant murine IL-4 (10 ng/ml) and IL-13 (10 ng/ml) were added to cell culture to induce BMDM immunosuppressive polarization. At day 7, transduced BMDMs were collected for flow cytometry analysis of transduction efficiency (% GFP⁺ cells of total cells): GFP⁺ BMDMs were sorted via FACS using a FACSAria II flow cytometer (BD Biosciences) and were then used for QPCR analysis of immunosuppressive gene expression.

### Human Monocyte-Derived Macrophage (MDM) Culture and Polarization

Human peripheral blood mononuclear cells (PBMCs) of healthy donors were obtained from the CFAR Gene and Cellular Therapy Core Laboratory at UCLA, without identification information under federal and state regulations. Human monocytes were isolated from healthy donor PBMCs by adherence. Briefly, PBMCs were suspended in serum-free RPMI 1640 media (Corning Cellgro, 10-040-CV) at 10×10⁶ cells/ml. 12.5 ml of the cell suspension were added to each 10-cm dish and incubated for one hour in a humidified 37° C., 5% CO₂ incubator. Medium that contained non-adherent cells was discarded. Dishes were washed twice and adherent monocytes were cultured in C10 media with human M-CSF (10 ng/ml) (Peprotech, 300-25) for 6 days to generate MDMs. At day 6, the resulting MDMs were collected and reseeded in a 6-well plate in C10 medium (1×10⁶ cells/2 ml/well) for 48 hours, in the presence or absence of recombinant human IL-4 (10 ng/ml) (Peprotech, 214-14) and human IL-13 (10 ng/ml) (Peprotech, 214-13) to induce MDM immunosuppressive polarization. In some experiments, MAOIs (phenelzine, 20 μM) were added to the MDM polarization culture 30 minutes prior to adding recombinant human IL-4 and human IL-13, to block MAO-A activity during MDM polarization. Polarized MDMs were then collected and used for flow cytometry and QPCR analysis or for setting up the 3D human tumour organoid culture experiments.

### Human NY-ESO-1-Specific TCR-Engineered CD8+ T (ESO-T) Cells

The Retro/ESO-TCR vector was constructed by inserting into the parental pMSGV vector a synthetic gene encoding an HLA-A2-restricted, NY-ESO-I tumour antigen-specific human CD8 TCR (clone 3A1)⁹⁸. Vsv-g-pseudotyped Retro/ESO-TCR retroviruses were generated by transfecting HEK 293T cells following a standard calcium precipitation protocol and an ultracentrifugation concentration protocol¹⁰²; the viruses were then used to transduce PG13 cells to generate a stable retroviral packaging cell line producing GALV-pseudotyped Retro/ESO-TCR retroviruses (denoted as the PG13-ESO-TCR cell line). For virus production, the PG13-ESO-TCR cells were seeded at a density of 0.8×10⁶ cells per ml in D10 medium, and cultured in a 15-cm dish (30 ml per dish) for 2 days: virus supernatants were then harvested and stored at −80° C. for future use.

Healthy donor PBMCs were cultured in a 12-well plate in CIO medium (1 x cells/ml/well) for 2 days, stimulated with Dynabeads™ Human T-Activator CD3/CD28 (10 μl/ml) (GIBCO, 11161D) and recombinant human IL-2 (20 ng/ml) (Peprotech). After 2 days, dynabeads were removed and cells were spin-infected with frozen-thawed Retro/ESO-TCR retroviral supernatants supplemented with polybrene (10 μg/ml) at 660 g at 30° C. for 90 minutes following an established protocol⁹⁸. Transduced human CD8⁺ T cells (denoted as ESO-T cells) were expanded for another 6-8 days in C10 medium containing recombinant human IL-2 (20 ng/ml) (Peprotech), and then cryopreserved for future use. Mock-transduced human CD8⁺ T cells (denoted as Mock-T cells) were generated as controls.

### 3D Human Tumour/TAM/T Cell Organoid Culture

A375-A2-ESO human melanoma cell line was generated by engineering the parental A375 cell line to overexpress an NY-ESO-1 tumour antigen as well as its matching HLA-A2 molecule⁹⁸. Human MDMs were generated from healthy donor PBMCs and polarized with IL-4/IL-13 in the presence or absence of phenelzine treatment. ESO-T cells were generated by engineering healthy donor PBMC CD8⁺ T cells to express an NY-ESO-I-specific TCR (clone 3A1). The A375-A2-ESO tumour cells, MDMs, and ESO-T cells were mixed at a 2:1:2 ratio. Mixed cells were centrifuged and resuspended in C10 medium at 1×10⁵ cells per μl medium. The cell slurry was adjusted to 5 ul per aggregate and was gently transferred onto a microporous membrane cell insert (Millicell, PICM0RG50) using a 20-μl pipet to form a 3D human tumour/TAM/T cell organoid. Prior to cell transfer, cell inserts were placed in a 6-well plate immersed with 1 ml CIO medium. Two days later, the organoids were dissociated by P1000 pipet tip and disrupted through a 70-μm nylon strainer to generate single cell suspensions for further analysis.

### Reagents

Adherent cell line culture medium (denoted as D10 medium) was made of Dulbecco's modified Eagle's medium (DMEM, Corning Cellgro, 10-013-CV) supplemented with 10% fetal bovine serum (FBS, Sigma-Aldrich, F2442) and 1% Penicillin-Streptomycin-Glutamine (Gibco, 10378016). T cell and macrophage culture medium (denoted as C10 medium) was made of RPMI 1640 (Corning Cellgro, 10-040-CV) supplemented with 10% FBS (Sigma-Aldrich), 1% Penicillin-Streptomycin-Glutamine (Gibco), 0.2% Normocin (Invivogen, ant-nr-2), 1% MEM Non-Essential Amino Acids Solution (Gibco, 11140050), 1% HEPES (Gibco, 15630056), and 1% Sodium Pyruvate (Gibco, 11360070).

Macrophage culture reagents, including recombinant murine IL-4, recombinant murine IL-13, recombinant human M-CSF, recombinant human IL-4, and recombinant human IL-13 were purchased from PeproTech. T cell culture reagents, including purified NA/LE anti-mouse CD3ε (clone 145-2C 11), anti-mouse CD28 (clone 37.51), anti-human CD3 (clone OKT3), and anti-human CD28 (clone CD28.2), were purchased from BD Biosciences. Recombinant human IL-2 was purchased from PeproTech. Hydrogen peroxide solution was purchased from Sigma-Aldrich (216763).

In vivo PD-1 blocking antibody (clone RMP1-14) and its isotype control (rat IgG2a) were purchased from BioXCell. In vivo TAM depletion clodronate liposomes and their control vehicle liposomes were purchased from Clodrosome.

Monoamine oxidase inhibitors (MAOIs), including phenelzine, moclobimide, and clorgyline, were purchased from Sigma-Aldrich. Pirlindole was purchased from R&D systems.

Detailed reagent information is provided in Table 2.

### Flow Cytometry

Flow cytometry, also known as FACS (fluorescence-activated cell sorting), was used to analyze surface marker and intracellular effector molecule expression in immune cells. Fluorochrome-conjugated monoclonal antibodies specific for mouse CD45.2 (clone 104), CDI11b (Clone M1/70), Ly6G (Clone 1A8), F4/80 (Clone BM8), Ly6C (Clone HK1.4), CD206 (Clone C068C2), CD69 (clone H1.2F3), CD86 (Clone GL-1), I-Ab (Clone AF6-120.1), TCRβ (clone H57-597), CD45.1 (Clone A20), CD4 (Clone GK1.5), CD8 (clone 53-6.7), CD25 (clone PC61), CD44 (clone IM7), CD62L (clone MEL-14), and Granzyme B (Clone QA16A02) were purchased from BioLegend. Mouse Fc Block (anti-mouse CD16/32: clone 2.4G2) was purchased from BD Biosciences. Fluorochrome-conjugated monoclonal antibodies specific for human CD45 (clone H130), CD11b (Clone ICRF44), CD14 (Clone HCD14), CD206 (Clone 15-2), CD273 (Clone 24F.10C12), TCRαβ (clone 126), CD4 (clone OKT4), CD8 (clone SK1), CD44 (clone IM7), CD62L (clone DREG-56), and human Fc Receptor Blocking Solution (TruStain FcX™, 422302) were purchased from BioLegend. Fixable Viability Dye eFluor 506 was purchased from Thermo Fisher Scientific. DAPI (Thermo Fisher Scientific) was included to exclude dead cells in FACS sorting.

To study cell surface marker expression, cells were stained with Fixable Viability Dye followed by Fc blocking and surface marker staining, following a standard procedure as described previously¹⁰¹. To study T cell intracellular cytotoxicity molecule production, intracellular staining of Granzyme B was performed using the BD Cytofix/Cytoperm™ Fixation/Permeabilization Kit (BD Biosciences, 55474) following the manufacturer's instructions. These cells were co-stained with surface markers to identify CD8⁺ T cells (gated as TCRβ⁺CD8⁺ cells in vitro or CD45.2⁺TCRβ⁺CD8⁺ cells in vivo).

Stained cells were analyzed using a MACSQuant Analyzer 10 flow cytometer (Miltenyi Biotec): data were analyzed using a FlowJo software (BD Biosciences).

Detailed reagent and resources information is provided in Table 2.

### Western Blot (WB)

Total protein was extracted using a RIPA lysis buffer (PIERCE, Roche, Thermo Fisher Scientific) supplemented with protease inhibitor cocktail cOmplete Mini (1 tablet/10 ml) (Sigma-Aldrich, 4693159001) and phosphotase inhibitor PhosSTOP (1 tablet/10 ml) (Sigma-Aldrich, 4906845001), then transferred to pre-cooled eppendorf tubes. The lysed solution was kept on ice for 30 minutes, and then centrifuged at 15,000 g for 5 minutes at 4° C. Supernatants were collected and protein concentrations were quantified using a BCA protein assay (PIERCE, Thermo Fisher Scientific, 23225). Equal amounts of protein were loaded and separated by 8% sodium dodecyl sulfate-polyacrylamide gel electrophoresis (SDS-PAGE), and then transferred to an Immunobilon-P PVDF Membrane (Millipore). The membranes were blocked with a SuperBlock™ T20 (TBS) Blocking Buffer (Thermo Fisher Scientific, 37536). Antibodies were diluted in 5% nonfat milk dissolved in washing buffer TBST (20 mM Tris-HCl, 150 mM NaCl, 0.1% Tween-20).

Primary antibodies against mouse Stat6, p-Stat6 (Tyr641), JAK1, p-JAK1 (Tyr1034/1035), JAK2, p-JAK2 (Tyr1008), JAK3, p-JAK3 (Tyr980/981), HRP-labeled anti-rabbit secondary antibodies, and HRP-labeled anti-mouse secondary antibodies were purchased from Cell Signaling Technology. MAO-A antibody was purchased from Abcam (Clone EPR7101). Primary antibodies against β-actin (Santa Cruz Biotechnology) were used as an internal control for total protein extracts. Signals were visualized using a ChemiDoc Image System (Bio-Rad). Data were analyzed using an Image J software (Bio-Rad).

Detailed reagent information is provided in Table 2.

### Quantitative Real-Time PCR (QPCR)

Total RNA was extracted from cells using TRIzol reagent (Invitrogen, Thermo Fisher Scientific, 15596018) following the manufacturer's instructions. SuperScript III First-strand (Thermo Fisher Scientific, 18080051) was used for reverse transcription. QPCR was performed using a KAPA SYBR FAST qPCR Master Mix (Kapa Biosystems) and a 7500 Real-time PCR System (Applied Biosystems) according to the manufacturers' instructions. Housekeeping gene Ube2d2 was used as an internal control for mouse immune cells and ACTB was used as an internal control for human immune cells. The relative expression of a target gene was calculated using the ΔΔCT method.

### Reactive Oxygen Species (ROS) Measurement

Cells were stained with surface marker antibodies, washed with PBS, then resuspended in pre-warmed PBS (1×10⁶ cells/ml/tube) containing 1 μM CM-H2DCFDA (Thermo Fisher Scientific, C6827). After 15 minutes incubation at room temperature, cells were immediately washed with cold PBS followed by flow cytometry analysis. ROS levels were measured by oxidation of the CM-H2DCFDA probes that can be read out as the fluorescence intensity at the FITC/488 channel of a flow cytometer.
Single cell RNA sequencing (scRNAseq)
scRNASeq was used to analyze the gene expression profiles of TIIs. B16-OVA tumours were harvested from Maoa WT and Maoa KO mice to prepare TII suspensions (10 tumours were combined for each group). TII suspensions were then sorted using a FACSAria II flow cytometer to purify immune cells (gated as DAPI-CD45.2⁺ cells). Sorted TIIs were immediately delivered to the Technology Center for Genomics & Bioinformatics (TCGB) facility at UCLA for library construction and sequencing. Cells were stained with trypan blue (Thermo Fisher Scientific, T10282) and counted using a Cell Countess II automated cell counter (Thermo Fisher Scientific). 10,000 TIIs from each experimental group were loaded on the Chromium platform (10X Genomics) and libraries were constructed using a Chromium Single Cell 3′ library & Gel Bead Kit V2 (10X Genomics, PN-120237) according to the manufacturer's instructions. Libraries were sequenced on an Illumina Novaseq 6000 System, using a Novaseq 6000 S2 Reagent Kit (100 cycles: 20012862, Illumina). Data analysis was performed using a Cellranger Software Suite (10X Genomics). BCL files were extracted from the sequencer and used as inputs for the cellranger pipeline to generate the digital expression matrix for each sample. Then cellranger aggr command was used to aggregate the two samples into one digital expression matrix. The matrix was analyzed using Seurat, an R package designed for single cell RNA sequencing. Specifically, cells were first filtered to have at least 300 UMIs (unique molecular identifiers), at least 100 genes and at most 50% mitochondrial gene expression: only 1 cell did not pass the filter. The filtered matrix was normalized using the Seurat function NormalizeData. Variable genes were found using the Seurat function FindVariableGenes. The matrix was scaled to regress out the sequencing depth for each cell. Variable genes that had been previously identified were used in principle component analysis (PCA) to reduce the dimensions of the data. Following this, 13 PCs were used in UMAP to further reduce the dimensions to 2. The same 13 PCs were also used to group the cells into different clusters by the Seurat function FindClusters. Next, marker genes were found for each cluster and used to define the cell types. Subsequently, 2 clusters of TAMs (identified by co-expression of Mrc1 and Cd86 signature genes) were extracted and compared between the Maoa WT and Maoa KO samples. Expression distribution of immunosuppressive and immunostimulatory signature genes in Maoa WT and Maoa KO TAMs were compared and presented in violin plots.

### Tumour Immune Dysfunction and Exclusion (TIDE) Computational Method

TIDE analyses were conducted as previously described (http://tide.dfci.harvard.edu)⁷⁵. Two functions of the TIDE computational method were used: 1) the prioritization function and 2) the survival correlation function.

The prioritization function of TIDE was used to rank a target gene by its immune dysfunction/risk score, that for TAMs was calculated as its gene expression log-fold change of M2-like/M1-like MDMs⁷⁵. A transcriptome data set (GSE35449) was used, which was generated by microarray analysis of the gene expression profiling of in vitro polarized MI-like or M2-like human MDMs⁷⁶. A score higher than 1 indicates the preferential expression of a gene in M2-like compared to MI-like human macrophages. The higher a score is, the more “prioritized” a gene is in relating to TAM immunosuppressive polarization.

The survival correlation function of TIDE was used to study the clinical data correlation between the intratumoural MAOA gene expression and patient survival. Four patient cohorts were analyzed: ovarian cancer (GSE26712)⁷⁸, lymphoma (GSE10846)⁷⁹, breast cancer (GSE9893)⁸⁰, and melanoma (PRJEB23709)⁸¹. For each patient cohort, tumour samples were divided into two groups: MAOA-high (samples with MAOA expression one standard deviation above the average) and MAOA-low (remaining samples) groups. The association between the intratumoural MAOA gene expression levels and patient overall survival (OS) was computed through the two-sided Wald test in the Cox-PH regression and presented in Kaplan-Meier plots. P value indicates the comparison between the MAOA-low and MAOA-high groups, and was calculated by two-sided Wald test in a Cox-PH regression.

### Statistical Analysis

GraphPad Prism 6 (GraphPad Software) was used for the graphic representation and statistical analysis of the data. All data were presented as the mean±standard error of the mean (SEM). A 2-tailed Student's t test was used for comparison between groups. Multiple comparisons were performed using an ordinary 1-way ANOVA followed by Tukey's multiple comparisons test, or using a 2-way ANOVA followed by Sidak's multiple comparisons test. P<0.05 was considered statistically significant. ns, not significant. *P<0.05; **P<0.01; ***P<0.001. For scRNAseq data analysis, Wilcoxon-rank sum test was utilized to determine the P value between two groups. Benjamini-Hochberg Procedure was used to adjust the P value to reduce the false positive rate. For the Kaplan-Meier plot of the overall patient survival for ovarian cancer, lymphoma, breast cancer, and melanoma with different MAOA levels, the P value was calculated by two-sided Wald test in a Cox-PH regression.

TABLE 1
Patient information for the ovarian cancer tumour samples.

| Date | | | Tumour |
|---|---|---|---|
| collected | Age | Diagnosis | provided |
| Mar. 26, | 55 | Stage IIIC ovarian cancer (high grade | Ovarian |
| 2019 | | serous adenocarcinoma type), status | tumour |
| | | post neoadjuvant chemotherapy (3 cycles | |
| | | Carboplatin/Taxol) | |
| Jul. 1, | 54 | Synchronous stage IA ovarian | Adnexal |
| 2019 | | adenocarcinoma and stage IA uterine | tumour |
| | | adenocarcinoma (endometrioid type, FIGO | |
| | | grade 1) | |
| Jan. 20, | 61 | Dedifferentiated carcinoma, FIGO grade | Ovarian |
| 2020 | | III | tumour |
| Feb. 13, | 75 | High grade serous ovarian carcinoma, | Omental |
| 2020 | | stage IIIC | tumour |

TABLE 2
Reagent and resources information.

| | | |
|---|---|---|
| REAGENT or RESOURCE | SOURCE | IDENTIFIER |

Antibodies

| | | |
|---|---|---|
| Purified Anti-Mouse CD16/CD32 (Mouse Fc | BD | CAT#553142; RRID: |
| Block) | Biosciences | AB_394657 |
| Anti-mouse CD4 Antibody (Clone GK1.5) | Biolegend | CAT#100428; RRID: |
| | | AB_493647 |
| Anti-mouse CD8a Antibody (Clone 53-6.7) | Biolegend | CAT#100732; RRID: |
| | | AB_893423 |
| Anti-mouse CD25 Antibody (Clone PC61) | Biolegend | CAT#102008; RRID: |
| | | AB_312857 |
| Anti-mouse TCR β chain Antibody (Clone | Biolegend | CAT#109220; RRID: |
| H57-597) | | AB_893624 |
| Anti-mouse/human CD44 Antibody (Clone | Biolegend | CAT#103006; RRID: |
| IM7) | | AB_312957 |
| Anti-mouse CD62L Antibody (Clone | Biolegend | CAT#104412; RRID: |
| MEL-14) | | AB_313099 |
| Anti-mouse CD45.2 Antibody (Clone 104) | Biolegend | CAT#109830; RRID: |
| | | AB_1186098 |
| Anti-mouse CD45.1 Antibody (Clone A20) | Biolegend | CAT#110716; RRID: |
| | | AB_313505 |
| Anti-human/mouse Granzyme B | Biolegend | CAT#372208; RRID: |
| Recombinant Antibody (Clone QA16A02) | | AB_2687032 |
| Anti-mouse F4/80 Antibody (Clone BM8) | Biolegend | CAT#123126; RRID: |
| | | AB_893483 |
| Anti-mouse/human CD11b Antibody (Clone | Biolegend | CAT#101206; RRID: |
| M1/70) | | AB_312789 |
| Anti-mouse Ly-6C Antibody (Clone HK1.4) | Biolegend | CAT#128018; RRID: |
| | | AB_1732082 |
| Anti-mouse CD69 Antibody (Clone H1.2F3) | Biolegend | CAT#104508; RRID: |
| | | AB_313111 |
| Anti-mouse I-Ab Antibody (Clone AF6- | Biolegend | CAT#116420; RRID: |
| 120.1) | | AB_10575296 |
| Anti-mouse CD86 Antibody (Clone GL-1) | Biolegend | CAT#105012; RRID: |
| | | AB_493342 |
| Anti-mouse Ly-6G Antibody (Clone 1A8) | Biolegend | CAT#127612; RRID: |
| | | AB_2251161 |
| Anti-mouse CD206 (MMR) Antibody | Biolegend | CAT#141720; RRID: |
| (Clone C068C2) | | AB_2562248 |
| Human Fc Receptor Blocking Solution | Biolegend | CAT#422302 |
| (TrueStain FcX) | | |
| Anti-human TCR(alpha)(beta) (Clone I26) | Biolegend | CAT#306716, RRID: |
| | | AB_1953257 |
| Anti-human CD45 (Clone H130) | Biolegend | CAT#304026, RFID: |
| | | AB_893337 |
| Anti-human CD4 (Clone OKT4) | Biolegend | CAT#317414, RRID: |
| | | AB_571959 |
| Anti-human CD8 (Clone SK1) | Biolegend | CAT#344714, RRID: |
| | | AB_2044006 |
| Anti-human CD14 (Clone HCD14) | Biolegend | CAT#325608, RRID: |
| | | AB_830681 |
| Anti-human CD11b (Clone ICRF44) | Biolegend | CAT#301330, RRID: |
| | | AB_2561703 |
| Anti-human CD206 (MMR) Antibody | Biolegend | CAT#321110, RRID: |
| (Clone 15-2) | | AB_571885 |
| Anti-human CD62L Antibody (Clone | Biolegend | CAT#304810, RRID: |
| DREG-56) | | AB_314470 |
| Anti-human CD273 (B7-DC, PD-L2) | Biolegend | CAT#329606, RRID: |
| Antibody (Clone 24F.10C12) | | AB_1089019 |
| Rat IgG2b, κ isotype control antibody (Clone | eBioscience | CAT#17-4031-82, RRID: |
| eB149/10H5) | | AB_470176 |
| Mouse IgG2b, κ isotype control antibody | Biolegend | CAT#400320 |
| (Clone MPC-11) | | |
| Jak1 (6G4) Rabbit mAb | Cell Signaling | CAT#3344 |
| Phospho-Jak1(Tyr1034/1035) (D7N4Z) | Cell Signaling | CAT#74129 |
| Rabbit mAb | | |
| Jak2 (D2E12) XP ®) Rabbit mAb | Cell Signaling | CAT#3230 |
| Phospho-Jak2 (Tyr1008) (D4A8) Rabbit | Cell Signaling | CAT#8082 |
| mAb | | |
| Jak3 (D7B12) Rabbit mAb | Cell Signaling | CAT#8863 |
| Phospho-Jak3 (Tyr980/981) (D44E3) Rabbit | Cell Signaling | CAT#5031 |
| mAb | | |
| Anti-rabbit IgG, HRP-linked Antibody | Cell Signaling | CAT#7074 |
| Rabbit Anti-Monoamine Oxidase A/MAO-A | Abcam | CAT#ab126751 |
| antibody (EPR7101) | | |
| β-Actin Antibody (AC-15) | Santa Cruz | CAT#sc-69879 |
| Anti-mouse IgG, HRP-linked Antibody | Cell Signaling | CAT#7076 |
| aPD-1 blocking antibody (clone RMP1-14) | BioXCell | CAT#E0146 |
| aPD-l isotype control (rat IgG2a) | BioXCell | CAT#BE0089 |

Bacterial and Virus Strains

| | | |
|---|---|---|
| Lenti/HLA-A2 | This paper | N/A |
| Lenti/NY-ESO-1 | This paper | N/A |
| Retro/Maoa | This paper | N/A |
| Retro/ESO-TCR | This paper | N/A |

Biological Samples

| | | |
|---|---|---|
| Human peripheral blood mononuclear cells | UCLA | N/A |
| (hPBMCs) | | |
| Human ovarian cancer tissues | UCLA | N/A |

Chemicals, Peptides, and Recombinant Proteins

| | | |
|---|---|---|
| Recombinant human IL-4 | Peprotech | CAT#200-04 |
| Recombinant human IL-13 | Peprotech | CAT#200-13 |
| Recombinant human M-CSF | Peprotech | CAT#300-25 |
| Recombinant murine IL-4 | Peprotech | CAT#214-14 |
| Recombinant murine IL-13 | Peprotech | CAT#210-13 |
| CM-H₂DCFDA | Thermo Fisher | CAT#C6827 |
| | Scientific | |
| RPMI1640 cell culture medium | Corning | CAT#10-040-CV |
| | Cellgro | |
| DMEM cell culture medium | Corning | CAT#10-013-CV |
| | Cellgro | |
| Fetal Bovine Serum (FBS) | Sigma-Aldrich | CAT#F2442 |
| Penicillin-Streptomycine-Glutamine (P/S/G) | GIBCO | CAT#10378016 |
| MEM non-essential amino acids (NEAA) | GIBCO | CAT#11140050 |
| HEPES Buffer Solution | GIBCO | CAT#15630056 |
| Sodium Pyruvate | GIBCO | CAT#11360070 |
| Normocin | Invivogen | CAT#ant-nr-2 |
| Hydrogen peroxide solution | Sigma-Aldrich | CAT#216763 |
| Fixable Viability Dye eFluor506 | Affymetrix | CAT#65-0866-14 |
| | eBioscience | |
| Isoflurane | Zoetis | CAT#50019100 |
| Phosphate Buffered Saline (PBS) pH 7.4 | GIBCO | CAT#10010-023 |
| (1X) | | |
| Phorbol-12-myristate-13-acetate (PMA) | Calbiochem | CAT#524400 |
| Ionomycin, Calcium salt, Streptomyces | Calbiochem | CAT#407952 |
| conglobatus | | |
| SuperBlock ™ T20 (TBS) Blocking Buffer | Thermo Fisher | CAT#37536 |
| | Scientific | |
| Tyramine | Sigma-Aldrich | CAT#T90344 |
| Trypan blue | Thermo Fisher | CAT#T10282 |
| | Scientific | |

Critical Commercial Assays

| | | |
|---|---|---|
| Trizol | Invitrogen | CAT#15596018 |
| Human CD14 Microbeads | Miltenyi Biotec | CAT#130-050-201 |
| Dynabeads ™ Human T-Activator | Gibco | CAT#11161D |
| CD3/CD28 | | |
| Fixation/Permeabilization Solution Kit | BD | CAT#55474 |
| | Biosciences | |
| 70-μm cell strainer | Corning | CAT#07-201-431 |
| Percoll | Sigma-Aldrich | CAT#P4937 |
| Millicell Cell Culture Insert | Millicell | CAT#PICM0RG50 |
| cOmplete Mini | Sigma-Aldrich | CAT#4693159001 |

Experimental Models: Cell Lines

| | | |
|---|---|---|
| Human melanoma cell line A375 | ATCC | N/A |
| Human melanoma cell line A375-A2-ESO | This paper | N/A |
| Mouse melanoma cell line B16-OVA | Dr, Pin Wang | N/A |
| Mouse colon cancer cell line MC38 | Dr. Antoni | N/A |
| | Ribaz | |
| HEK 293T cell line | ATCC | N/A |
| PG13 cell line | Dr, Pin Wang | N/A |
| PG13-ESO-TCR cell line | This paper | N/A |
| Phoenix-ECO cell line | ATCC | N/A |
| L929 | ATCC | N/A |

Experimental Models: Organisms/Strains

| | | |
|---|---|---|
| Mouse: C57BL/6J (B6) | The Jackson | JAX: 000664 |
| | Laboratory | |
| Mouse: B6.SJL-Ptprc^{a}Pepc^{b}/BoyJ | The Jackson | JAX: 002014 |
| | Laboratory | |
| Mouse: 129S-Maoa^{tm1Shih}/J | The Jackson | JAX: 017340 |
| | Laboratory | |
| Mouse: NOD.Cg-Prkdc^{scid} Il2rg^{tm1Wjl}/SzJ | The Jackson | JAX: 005557 |
| (NSG) | Laboratory | |

Software and Algorithms

| | | |
|---|---|---|
| FlowJo Software | FlowJo | https://www.flowjo.com/ |
| | | solutions/flowjo/downloads |
| Prism 6 | Graphpad | https://www.graphpad.com/ |
| | | scientificsoftware/prism/ |
| I-control 1.7 Microplate Reader Software | Tecan | https://www.selectscience. |
| | | net/tecan/icontrol- |
| | | microplate-reader- |
| | | software/81307 |
| Aura imaging software | Spectral | https://specimg.com/aura/ |
| | Instruments | |
| | Imaging | |

1. Jenkins, R. W., Thummalapalli, R., Carter, J., Canadas, I. & Barbie, D. A. Molecular and Genomic Determinants of Response to Immune Checkpoint Inhibition in Cancer. Annu Rev Med 69, 333-347 (2018).
2. Ribas, A. Releasing the Brakes on Cancer Immunotherapy. N Engl J Med 373, 1490-1492(2015).
3. Page, D. B., Postow, M. A., Callahan, M. K., Allison, J. P. & Wolchok, J. D. Immune modulation in cancer with antibodies. Annu Rev Med 65, 185-202 (2014).
4. Hodi, F. S. et al. Improved survival with ipilimumab in patients with metastatic melanoma. N Engl J Med 363, 711-723 (2010).
5. Topalian, S. L. et al. Safety, activity, and immune correlates of anti-PD-1 antibody in cancer. N Engl J Med 366, 2443-2454 (2012).
6. Dougan, M., Dranoff, G. & Dougan, S. K. Cancer Immunotherapy: Beyond Checkpoint Blockade. Annu Rev Canc Biol 3, 55-75 (2019).
7. Sharpe, A. H. Introduction to checkpoint inhibitors and cancer immunotherapy. Immunol Rev 276, 5-8 (2017).
8. Li, Y. et al. A Mini-Review for Cancer Immunotherapy: Molecular Understanding of PD-1/PD-L1 Pathway & amp: Translational Blockade of Immune Checkpoints. Int J Mol Sci 17 (2016).
9. Topalian, S. L., Taube, J. M., Anders, R. A. & Pardoll, D. M. Mechanism-driven biomarkers to guide immune checkpoint blockade in cancer therapy. Nat Rev Cancer 16, 275-287 (2016).
10. Baumeister, S. H., Freeman, G. J., Dranoff, G. & Sharpe, A. H. Coinhibitory Pathways in Immunotherapy for Cancer. Annu Rev Immunol 34, 539-573 (2016).
11. Pardoll, D. M. The blockade of immune checkpoints in cancer immunotherapy. Nat Rev Cancer 12, 252-264 (2012).
12. Ascierto, P. A., Simeone, E., Sznol, M., Fu, Y. X. & Melero, I. Clinical experiences with anti-CD137 and anti-PD1 therapeutic antibodies. Semin Oncol 37, 508-516 (2010).
13. Sharpe, A. H. & Pauken, K. E. The diverse functions of the PD1 inhibitory pathway. Nat Rev Immunol 18, 153-167 (2018).
14. Peranzoni, E. et al. Macrophages impede CD8 T cells from reaching tumour cells and limit the efficacy of anti-PD-1 treatment. Proc Nall Acad Sci USA 115, E4041-E4050 (2018).
15. DeNardo, D. G. et al. Leukocyte complexity predicts breast cancer survival and functionally regulates response to chemotherapy. Cancer Discov 1, 54-67 (2011).
16. Cassetta, L. & Kitamura, T. Targeting Tumour-Associated Macrophages as a Potential Strategy to Enhance the Response to Immune Checkpoint Inhibitors. Front (Cell Dev Biol 6, 38 (2018).
17. Fujimura, T., Kambayashi, Y., Fujisawa, Y., Hidaka, T. & Aiba, S. Tumour-Associated Macrophages: Therapeutic Targets for Skin Cancer. Front Oncol 8, 3 (2018).
18. Awad, R. M., De Vlaeminck, Y., Maebe, J., Goyvaerts, C. & Breckpot, K. Turn Back the TIMe: Targeting Tumour Infiltrating Myeloid Cells to Revert Cancer Progression. Front Immunol 9, 1977 (2018).
19. Cannarile, M. A. et al. Colony-stimulating factor I receptor (CSF1R) inhibitors in cancer therapy. J Immunother Cancer 5, 53 (2017).
20. Liu, Y. & Cao, X. The origin and function of tumour-associated macrophages. Cell Mol Immunol 12, 1-4 (2015).
21. Wynn, T. A., Chawla, A. & Pollard, J. W. Macrophage biology in development, homeostasis and disease. Nature 496, 445-455 (2013).
22. Lavin, Y., Mortha, A., Rahman, A. & Merad, M. Regulation of macrophage development and function in peripheral tissues. Nat Rev Immunol 15, 731-744 (2015).
23. Biswas, S. K. Metabolic Reprogramming of Immune Cells in Cancer Progression. Immunity 43, 435-449 (2015).
24. Fujimura, T., Kakizaki, A., Furudate, S., Kambayashi, Y. & Aiba, S. Tumour-associated macrophages in skin: How to treat their heterogeneity and plasticity. J Dermatol Sci 83, 167-173 (2016).
25. Qian, B. Z. & Pollard, J. W. Macrophage diversity enhances tumour progression and metastasis. Cell 141, 39-51 (2010).
26. Jetten, N. et al. Anti-inflammatory M2, but not pro-inflammatory M1 macrophages promote angiogenesis in vivo. Angiogenesis 17, 109-118 (2014).
27. DeNardo, D. G. et al. CD4(+) T cells regulate pulmonary metastasis of mammary carcinomas by enhancing protumour properties of macrophages. Cancer Cell 16, 91-102 (2009).
28. Bordon, Y. Macrophages throw tumour cells a lifeline. Nat Rev Immunol 19, 202-203 (2019).
29. Coussens, L. M., Zitvogel, L. & Palucka, A. K. Neutralizing tumour-promoting chronic inflammation: a magic bullet? Science 339, 286-291 (2013).
30. Pollard, J. W. Trophic macrophages in development and disease. Nat Rev Immunol 9, 259-270 (2009).
31. Noy, R. & Pollard, J. W. Tumour-associated macrophages: from mechanisms to therapy. Immunity 41, 49-61 (2014).
32. Arlauckas, S. P. et al. Arg1 expression defines immunosuppressive subsets of tumour-associated macrophages. Theranostics 8, 5842-5854 (2018).
33. Caux, C., Ramos, R. N., Prendergast, G. C., Bendriss-Vermare, N. & Menetrier-Caux, C. A Milestone Review on How Macrophages Affect Tumour Growth. Cancer Res 76, 6439-6442 (2016).
34. Gabrilovich, D. I. & Nagaraj, S. Myeloid-derived suppressor cells as regulators of the immune system. Nat Rev Immunol 9, 162-174 (2009).
35. Geiger, R. et al. L-Arginine Modulates T Cell Metabolism and Enhances Survival and Anti-tumour Activity. Cell 167, 829-842 e813 (2016).
36. Schuette, V. et al. Mannose receptor induces T-cell tolerance via inhibition of CD45 and up-regulation of CTLA-4. Proc Natl Acad Sci USA 113, 10649-10654 (2016).
37. Biswas, S. K. & Mantovani, A. Macrophage plasticity and interaction with lymphocyte subsets: cancer as a paradigm. Nat Immunol 11, 889-896 (2010).
38. Bercovici, N., Guerin, M. V., Trautmann, A. & Donnadieu, E. The Remarkable Plasticity of Macrophages: A Chance to Fight Cancer. Front Immunol 10, 1563 (2019).
39. DeNardo, D. G. & Ruffell, B. Macrophages as regulators of tumour immunity and immunotherapy. Nat Rev Immunol 19, 369-382 (2019).
40. Ji, L. et al. Slc6a8-Mediated Creatine Uptake and Accumulation Reprogram Macrophage Polarization via Regulating Cytokine Responses. Immunity 51, 272-284 e277 (2019).
41. Jiang, M. et al. Tumour-targeted delivery of silibinin and IPI-549 synergistically inhibit breast cancer by remodeling the microenvironment. Int J Pharm 581, 119239 (2020).
42. Zhang, X., Shen, L., Liu, Q., Hou, L. & Huang, L. Inhibiting PI3 kinase-gamma in both myeloid and plasma cells remodels the suppressive tumour microenvironment in desmoplastic tumours. J Control Release 309, 173-180 (2019).
43. Tiihonen, J. et al. Genetic background of extreme violent behavior. Mol Psychiatry 20, 786-792 (2015).
44. Gibbons, A. American Association of Physical Anthropologists meeting. Tracking the evolutionary history of a “warrior” gene. Science 304, 818 (2004).
45. Shih, J. C., Chen, K. & Ridd, M. J. Monoamine oxidase: from genes to behavior. Annu Rev Neurosci 22, 197-217 (1999).
46. Brunner, H. G., Nelen, M., Breakefield, X. O., Ropers, H. H. & van Oost, B. A. Abnormal behavior associated with a point mutation in the structural gene for monoamine oxidase A. Science 262, 578-580 (1993).
47. Meyer, J. H. et al. Elevated monoamine oxidase a levels in the brain: an explanation for the monoamine imbalance of major depression. Arch Gen Psychiatry 63, 1209-1216 (2006).
48. Dias, V., Junn, E. & Mouradian, M. M. The role of oxidative stress in Parkinson's disease. J Parkinsons Dis 3, 461-491 (2013).
49. Tong, J. et al. Brain monoamine oxidase B and A in human parkinsonian dopamine deficiency disorders. Brain 140, 2460-2474 (2017).
50. Benson, C. A., Wong, G., Tenorio, G., Baker, G. B. & Kerr, B. J. The MAO inhibitor phenelzine can improve functional outcomes in mice with established clinical signs in experimental autoimmune encephalomyelitis (EAE). Behav Brain Res 252, 302-311 (2013).
51. Finberg, J. P. & Rabey, J. M. Inhibitors of MAO-A and MAO-B in Psychiatry and Neurology. Front Pharmacol 7, 340 (2016).
52. Musgrave, T. et al. The MAO inhibitor phenelzine improves functional outcomes in mice with experimental autoimmune encephalomyelitis (EAE). Brain Behav Immun 25, 1677-1688 (2011).
53. Bolasco, A., Carradori, S. & Fioravanti, R. Focusing on new monoamine oxidase inhibitors. Expert Opin Ther Pat 20, 909-939 (2010).
54. Bortolato, M., Chen, K. & Shih, J. C. Monoamine oxidase inactivation: from pathophysiology to therapeutics. Adv Drug Deliv Rev 60, 1527-1533 (2008).
55. Riederer, P. & Laux, G. MAO-inhibitors in Parkinson's Disease. Exp Neurobiol 20, 1-17 (2011).
56. Bortolato, M. et al. Social deficits and perseverative behaviors, but not overt aggression, in MAO-A hypomorphic mice. Neuropsychopharmacology 36, 2674-2688 (2011).
57. Zhang, Y. et al. ROS play a critical role in the differentiation of alternatively activated macrophages and the occurrence of tumour-associated macrophages. Cell Res 23, 898-914 (2013).
58. He, C., Ryan, A. J., Murthy, S. & Carter, A. B. Accelerated development of pulmonary fibrosis via Cu, Zn-superoxide dismutase-induced alternative activation of macrophages. J Biol Chem 288, 20745-20757 (2013).
59. Murthy, S., Ryan, A., He, C., Mallampalli, R. K. & Carter, A. B. Rac1-mediated mitochondrial H₂O₂ generation regulates MMP-9 gene expression in macrophages via inhibition of SP-1 and AP-1. J Biol Chem 285, 25062-25073 (2010).
60. Zhang, L. et al. Oxidative stress and asthma: proteome analysis of chitinase-like proteins and FIZZ1 in lung tissue and bronchoalveolar lavage fluid. J Proteome Res 8, 1631-1638 (2009).
61. Vats, D. et al. Oxidative metabolism and PGC-1beta attenuate macrophage-mediated inflammation. Cell Metab 4, 13-24 (2006).
62. Fu, C. et al. Activation of the IL-4/STAT6 Signaling Pathway Promotes Lung Cancer Progression by Increasing M2 Myeloid Cells. Front Immunol 10, 2638 (2019).
63. Van Dyken, S. J. & Locksley, R. M. Interleukin-4- and interleukin-13-mediated alternatively activated macrophages: roles in homeostasis and disease. Annu Rev Immunol 31, 317-343 (2013).
64. Bhattacharjee, A. et al. IL-4 and IL-13 employ discrete signaling pathways for target gene expression in alternatively activated monocytes/macrophages. Free Radic Biol Med 54, 1-16 (2013).
65. Nelms, K., Keegan, A. D., Zamorano, J., Ryan, J. J. & Paul, W. E. The IL-4 receptor: signaling mechanisms and biologic functions. Annu Rev Immunol 17, 701-738 (1999).
66. Dwivedi, G., Gran, M. A., Bagchi, P. & Kemp, M. L. Dynamic Redox Regulation of IL-4 Signaling. PLoS Comput Biol 11, e1004582 (2015).
67. Park, S. J. et al. Astrocytes, but not microglia, rapidly sense H(2)O(2)via STAT6 phosphorylation, resulting in cyclooxygenase-2 expression and prostaglandin release. J Immunol 188, 5132-5141 (2012).
68. Duhe, R. J. Redox regulation of Janus kinase: The elephant in the room. JAKSTAT 2, e26141 (2013).
69. Duan, W. et al. New role of JAK2/STAT3 signaling in endothelial cell oxidative stress injury and protective effect of melatonin. PLoS One 8, e57941 (2013).
70. Abe, J. & Berk, B. C. Fyn and JAK2 mediate Ras activation by reactive oxygen species. J Biol Chem 274, 21003-21010 (1999).
71. Simon, A. R., Rai, U., Fanburg, B. L. & Cochran, B. H. Activation of the JAK-STAT pathway by reactive oxygen species. Am J Physiol 275, C1640-1652 (1998).
72. Wimbiscus, M., Kostenko, O. & Malone, D. MAO inhibitors: risks, benefits, and lore. Cleve Clin J Med 77, 859-882 (2010).
73. Bruhwyler, J., Liegeois, J. F. & Geczy, J. Pirlindole: a selective reversible inhibitor of monoamine oxidase A. A review of its preclinical properties. Pharmacol Res 36, 23-33 (1997).
74. Homet Moreno, B. et al. Response to Programmed Cell Death-I Blockade in a Murine Melanoma Syngeneic Model Requires Costimulation, CD4, and CD8 T Cells. Cancer Immunol Res 4, 845-857 (2016).
75. Jiang, P. et al. Signatures of T cell dysfunction and exclusion predict cancer immunotherapy response. Nat Med 24, 1550-1558 (2018).
76. Beyer, M. et al. High-resolution transcriptome of human macrophages. PLoS One 7, e45466 (2012).
77. Thomas, R. et al. NY-ESO-1 Based Immunotherapy of Cancer: Current Perspectives. Front Immunol 9, 947 (2018).
78. Bonome, T. et al. A gene signature predicting for survival in suboptimally debulked patients with ovarian cancer. Cancer Res 68, 5478-5486 (2008).
79. Lenz, G. et al. Stromal gene signatures in large-B-cell lymphomas. N Engl J Med 359, 2313-2323 (2008).
80. Chanrion, M. et al. A gene expression signature that can predict the recurrence of tamoxifen-treated primary breast cancer. Clin Cancer Res 14, 1744-1752 (2008).
81. Gide, T. N. et al. Distinct Immune Cell Populations Define Response to Anti-PD-1 Monotherapy and Anti-PD-1/Anti-CTLA-4 Combined Therapy. Cancer Cell 35, 238-255 e236 (2019).
82. Talbot, S., Foster, S. L. & Woolf, C. J. Neuroimmunity: Physiology and Pathology. Annu Rev Immunol 34, 421-447 (2016).
83. Franco, R., Pacheco, R., Lluis, C., Ahem, G. P. & O'Connell, P. J. The emergence of neurotransmitters as immune modulators. Trends Immunol 28, 400-407 (2007).
84. Kerage, D., Sloan, E. K., Mattarollo, S. R. & McCombe, P. A. Interaction of neurotransmitters and neurochemicals with lymphocytes. J Neuroimmunol 332,99-111 (2019).
85. Yang, L. & Zhang, Y. Tumour-associated macrophages: from basic research to clinical application. J Hematol Oncol 10, 58 (2017).
86. Guerriero, J. L. et al. Class Ha HDAC inhibition reduces breast tumours and metastases through anti-tumour macrophages. Nature 543, 428-432 (2017).
87. Beatty, G. L. et al. CD40 agonists alter tumour stroma and show efficacy against pancreatic carcinoma in mice and humans. Science 331, 1612-1616 (2011).
88. Kaneda, M. M. et al. PI3Kgamma is a molecular switch that controls immune suppression. Nature 539, 437-442 (2016).
89. Zhang, J. Q. et al. Macrophages and CD8(+) T Cells Mediate the Antitumour Efficacy of Combined CD40 Ligation and Imatinib Therapy in Gastrointestinal Stromal Tumours. Cancer Immunol Res 6, 434-447 (2018).
90. Falkenberg, K. J. & Johnstone, R. W. Histone deacetylases and their inhibitors in cancer, neurological diseases and immune disorders. Nat Rev Drug Discov 13, 673-691 (2014).
91. Ghizzoni, M., Haisma, H. J., Maarsingh, H. & Dekker, F. J. Histone acetyltransferases are crucial regulators in NF-kappaB mediated inflammation. Drug Discov Today 16, 504-511 (2011).
92. Sai, J. et al. PI3K Inhibition Reduces Mammary Tumour Growth and Facilitates Antitumour Immunity and Anti-PDI Responses. Clin Cancer Res 23, 3371-3384 (2017).
93. Zheng, W. & Pollard, J. W. Inhibiting macrophage PI3Kgamma to enhance immunotherapy. Cell Res 26, 1267-1268 (2016).
94. Schein, C. H. Repurposing approved drugs on the pathway to novel therapies. Med Res Rev (2019).
95. Jessen, L., Kovalick, L. J. & Azzaro, A. J. The selegiline transdermal system (emsam): a therapeutic option for the treatment of major depressive disorder. P T 33, 212-246 (2008).
96. Shoval, G. et al. Adherence to antidepressant medications is associated with reduced premature mortality in patients with cancer: A nationwide cohort study. Depress Anxiety 36, 921-929 (2019).
97. Liu, Y. el al. In situ modulation of dendritic cells by injectable thermosensitive hydrogels for cancer vaccines in mice. Biomacromolecules 15, 3836-3845 (2014).
98. Bethune, M. T. el al. Isolation and characterization of NY-ESO-1-specific T cell receptors restricted on various MHC molecules. Proc Natl Acad Sci USA 115, E10702-E10711 (2018).
99. Di Biase, S. el al. Creatine uptake regulates CD8 T cell antitumour immunity. J Exp Med 216, 2869-2882 (2019).
100. Smith, D. J. el al. Genetic engineering of hematopoietic stem cells to generate invariant natural killer T cells. Proc Natl Acad Sci USA 112, 1523-1528 (2015).
101. Li, B. el al. miR-146a modulates autoreactive Th17 cell differentiation and regulates organ-specific autoimmunity. J Clin Invest 127, 3702-3716 (2017).
102. Smith, D. J. el al. Propagating Humanized BLT Mice for the Study of Human Immunology and Immunotherapy. Stem Cells Dev 25, 1863-1873 (2016).

All publications mentioned herein (e.g. Wang et al., Targeting monoamine oxidase A for T cell-based cancer immunotherapy: Sci Immunol. 2021 May 14:6(59):eabh2383. doi: 10.1 126/sciimmunol.abh2383. PMID: 33990379 and Wang et al., Targeting monoamine oxidase A-regulated tumor-associated macrophage polarization for cancer immunotherapy: Nat Commun. 2021 Jun. 10:12(1):3530. doi: 10.1038/s41467-021-23164-2: and the references numerically listed above) are incorporated herein by reference to disclose and describe aspects, methods and/or materials in connection with the cited publications.

1. A composition of matter comprising:
a chemotherapeutic agent;
a monoamine oxidase A inhibitor; and
a pharmaceutically acceptable carrier.

2. The composition of claim 1, wherein a monoamine oxidase A inhibitor comprises at least one of:
phenelzine; moclobemide; clorgyline; pirlindole; isocarboxazid; tranylcypromide;
iproniazid; caroxazone; befloxatone; brofaromine; cimoxatone; eprobemide; esuprone;
metraindol; or toloxatone.

3. The composition of claim 1, wherein:
the composition comprises a lipid; and/or
the composition comprises the monoamine oxidase A inhibitor disposed within a nanoparticle.

4. The composition of claim 1, wherein a monoamine oxidase A inhibitor is present in the composition in amounts such that amounts of monoamine oxidase A inhibitor available for tumor-associated macrophages in an individual administered the composition are sufficient to modulate the phenotype of the tumor-associated macrophages.

5. The composition of claim 4, wherein modulation of the phenotype of the tumor-associated macrophages comprises at least one of: decreased levels of intracellular reactive oxygen species; enhanced tumor immunoreactivity; increased expression of CD69, CD86 or MHC class II I-ab; or decreased expression of CD206.

6. The composition of claim 1, wherein the chemotherapeutic agent comprises:
an antibody;
carboplatin;
paclitaxel; or
at least one immune checkpoint inhibitor selected to affect CTLA-4 or a PD-1/PD-L1 blockade.

6. (canceled)

7. The composition of claim 4, wherein the antibody comprises at least one of:
pembrolizumab;
nivolumab;
atezolizumab;
avelumab;
bevacizumab; and
durvalumab.

8. A method of modulating a phenotype of a tumor-associated macrophage comprising introducing a monoamine oxidase A inhibitor in the environment in which the CD8 T cell is disposed; wherein amounts of the monoamine oxidase A inhibitor introduced into the environment are selected to be sufficient to modulate the phenotype of the tumor-associated macrophage.

9. The method of claim 8, wherein the tumor-associated macrophage is disposed in an individual diagnosed with cancer.

10. The method of claim 9, wherein the individual is undergoing a therapeutic regimen comprising the administration of a chemotherapeutic agent.

11. The method of claim 9, wherein the cancer is a lymphoma or a skin, breast, ovarian, prostate, colorectal or lung cancer.

12. The method of claim 8, wherein modulation of the phenotype of the tumor-associated macrophage comprises at least one of: decreased levels of intracellular reactive oxygen species; enhanced tumor immunoreactivity; increased expression of CD69, CD86 or MHC class II I-ab; or decreased expression of CD206.

13. The method of claim 8, wherein the monoamine oxidase A inhibitor comprises at least one of:
phenelzine; moclobemide; clorgyline; pirlindole; isocarboxazid; tranylcypromide; iproniazid; caroxazone; befloxatone; brofaromine; cimoxatone; eprobemide; esuprone; metraindol; or toloxatone.

14. The method of claim 13, wherein the monoamine oxidase A inhibitor is disposed within a nanoparticle; optionally a nanoparticle comprising a lipid.

15. The method of claim 10, wherein the chemotherapeutic agent comprises:
an antibody;
carboplatin;
paclitaxel; or
at least one immune checkpoint inhibitor selected to affect CTLA-4 or a PD-1/PD-L1 blockade.

16. A method of treating a cancer in an individual comprising administering to the individual a monoamine oxidase A inhibitor; wherein amounts of the monoamine oxidase A inhibitor administered to the individual are selected to be sufficient to modulate the phenotype of tumor-associated macrophages in the individual.

17. The method of claim 16, wherein modulation of the phenotype of the tumor-associated macrophages comprises at least one of: decreased levels of intracellular reactive oxygen species; enhanced tumor immunoreactivity; increased expression of CD69, CD86 or MHC class II I-ab; or decreased expression of CD206.

18. The method of claim 16, wherein the individual is undergoing a therapeutic regimen comprising the administration of at least one chemotherapeutic agent.

19. The method of claim 16, wherein the cancer is a lymphoma or a skin, breast, ovarian, prostate, colorectal or lung cancer.

20. The method of claim 8, wherein the monoamine oxidase A inhibitor is disposed within a composition comprising a crosslinked multilamellar liposome having an exterior surface and an interior surface, the interior surface defining a central liposomal cavity, the multilamellar liposome including at least a first lipid bilayer and a second lipid bilayer, the first lipid bilayer being covalently bonded to the second lipid bilayer; and the monoamine oxidase A inhibitor disposed within the liposome.
